(19) United States
(12) Patent Application Publication
(10) Pub. No.: US 2026/0360164 A1
(43) Pub. Date: Nov. 12, 2026

(54) METHOD AND SYSTEM OF MULTI-MODAL SUB-CELLULAR SEGMENTATION

(71) Applicant: Bruker Spatial Biology, Inc., Seattle, WA (US)

(72) Inventors: Aster WARDHANI, Seattle, WA (US); Lidan WU, Seattle, WA (US); Dwayne DUNAWAY, Seattle, WA (US); Carl BROWN, Seattle, WA (US); Sanghamithra KORUKONDA, Seattle, WA (US); David ROSS, Seattle, WA (US); John E. CHANDLER, Seattle, WA (US); Winnie Wing-Yin LEUNG, Seattle, WA (US)

(73) Assignee: Bruker Spatial Biology, Inc., Seattle, WA (US)

(21) Appl. No.: 19/487,061

(22) PCT Filed: Jun. 12, 2024
(86) PCT No.: PCT/US2024/033630

§ 371 Date: Nov. 24, 2025

Related U.S. Application Data
(60) Provisional application No. 63/507,824, filed on Jun. 13, 2023.

(51) Int. Cl. G06V 20/69; G06T 7/00; G06T 7/11

(52) U.S. Cl. CPC G06V 20/695; G06T 7/0012; G06T 7/11; G06T 2207/10056; G06T 2207/20081; G06T 2207/30024

(57) ABSTRACT

Described herein are systems and methods of multi-modal subcellular segmentation using photo-cleavable biomarkers and/or transcriptomic readout density maps. The systems and methods provide an improvement in cell segmentation accuracy for nuclei, cytoplasm and membrane regions through the use of optical and bleaching corrections from multiple sources of photo-cleavable morphology markers in combination with high-quality 3D images acquired with high-dynamic range scanning, and spatial transcriptomic readout density maps.

### CROSS-REFERENCE

This application is a 371 National Stage application of PCT/US24/33630, filed Jun. 12, 2024, which claims the benefit of priority of U.S. Provisional Application No. 63/507,824, filed on Jun. 13, 2023, the contents of which are hereby incorporated by reference in their entirety.

### BACKGROUND

Cell segmentation is a critical step in many biological and medical analyses, it provides a crucial foundation for spatial analysis that leverages the spatial organization and characteristics of cells within tissue samples. By precisely segmenting cells, spatial relationships and patterns can be studied, aiding in understanding tissue organization, cellular communication, or disease progression. Existing cell segmentation methods often suffer from low accuracy and high dependency on certain experimental conditions and tissue types.

### SUMMARY

Cell segmentation is a vision and image processing of dividing an image or a digital representation of cells into individual regions or segments, each corresponding to a specific cell or cell component. Cell segmentation in spatial biology enables several specific analyses that leverage the spatial organization and characteristics of cells within tissue samples, including spatial transcriptomics, spatial profiling, spatial clustering and spatial interaction analysis, spatial single-cell analysis, spatial co-expression analysis, spatial visualization and data integration. However, the cell boundary information is noisy from the expression variability of the morphology markers used. In some cases, the expression level can be absent or saturated causing cells to be missed or incorrectly segmented. The lack of reliable labeling of cellular membranes is a common challenge for robust cell segmentation in current spatial transcriptomics assays.

Existing cell segmentation methods often use a small set of morphology stains, typically between a single channel nuclei image or up to 3-channel RGB imaging of nuclei and membrane/cytoplasm stains. This present invention relates to a novel method for segmenting cells from microscope-derived images of tissues. A photo-cleavable marker can provide an unlimited set of source input channels by the process of repeat re-staining and clearing of markers using UV illumination and chemical wash in the tissue. The additional number of morphology markers can dramatically increase the amount of information on the cell boundary presented in the tissue. This present invention also provides a new approach utilizing spatial density generated from the readout density of the spatial transcriptomics assays as the morphology images for cell soma and performing cell segmentation on the generated readout density map.

In one aspect, disclosed herein are systems comprising at least one processor and instructions executable by the at least one processor to provide a multi-modal segmentation application comprising: (a) a software module configured to retrieve a 3D scan image of a biological sample in High Dynamic Range (HDR) mode, wherein the biological sample is labeled with one of a plurality of morphology markers; (b) a software module configured to reduce the 3D scan image to a 2D image, the optimal focus region in z-slice is obtained; or utilize the entire 3D stack volume (c) a software module configured to retrieve a readout density map from transcriptomic assays of the biological sample; and (d) a software module configured to perform subcellular segmentation on the biological sample based on the plurality of morphology markers or the readout density map. In some embodiments, the morphology markers comprise fluorescent dyes, nuclear stains, fluorescently labeled antibodies, immunohistochemistry (IHC) stains, photo-cleavable morphology markers, genetically encoded tags, magnetic resonance image (MRI) contrast agents, or nucleic acid probes. In some embodiments, the images are microscope-derived images, comprising images from optical microscopes, electron microscopes, or scanning probe microscopes. In some embodiments, the images can comprise images from spatial molecular imagers. In some embodiments, the images are applied to a bleaching correction. In some embodiments, the transcriptomic assays comprise gene expression assays with fluorescently labeled probes, RNA sequencing (RNA-seq), microarray analysis, reverse transcription polymerase chain reaction (RT-PCR), cap analysis of gene expression, or single-cell RNA sequencing (scRNA-seq).

In some embodiments, the software module is configured to retrieve at least 1 image, at least 3 images, at least 5 images, at least 10 images, at least 15 images, at least 20 images, at least 30 images, at least 35 images, at least 40 images, at least 45 images, at least 50 images, at least 55 images, at least 60 images, at least 65 images, at least 70 images, at least 80 images, at least 90 images, at least 100 images, at least 120 images, at least 150 images, at least 200 images, or more than at least 200 images of the biological sample. In some embodiments, the biological sample is obtained at least in part of one or more of biopsy collection, surgical resection, xenograft, animal model, fine needle aspiration, peripheral blood collection, bone marrow biopsy, healthy tissue sampling, neoplastic tissue sampling, malignant tissue sampling, diseased tissue sampling, and implanted tissue sampling. In some embodiments, the biological sample comprises cells or tissues. In some embodiments, the cells comprise primary cells, stem cells, immune cells, carcinoma cells, sarcoma cells, lymphoma cells, melanoma cells, cancer cells, or neoplastic cells. In some embodiments, subcellular segmentation comprises nucleic segmentation, cytoplasm segmentation, or extra-cellular segmentation. In some embodiments, subcellular segmentation comprises training a machine learning algorithm and/or applying a machine learning algorithm.

In another aspect, disclosed herein are non-transitory computer-readable storage media encoded with instructions executable by one or more processors to provide a multi-modal segmentation application comprising: (a) a software module configured to retrieve a 3D scan image of a biological sample in High Dynamic Range (HDR) mode, wherein the biological sample is labeled with one of a plurality of morphology markers; (b.1) optionally a software module configured to reduce the 3D scan image to a 2D image, retaining the optimal focus regions from various z slices, using techniques such as maximum intensity projection, focus stacking and extended depth of field; (b.2) alternatively a software module configured to deconvolve the 2D images to improve the sharpness and clarity of the images while maintaining the z position of those images in their 3D scan image stack (b.3) alternatively a software module configured to use each deconvolved 2D slice forming 3D volume stack directly for 3D cell segmentation; (c) a software module configured to retrieve a readout density map from transcriptomic assays of the biological sample; and (d) a software module configured to perform subcellular segmentation on the biological sample based on the plurality of morphology markers or the readout density map. In some embodiments, the morphology markers comprise fluorescent dyes, nuclear stains, fluorescently labeled antibodies, immunohistochemistry (IHC) stains, photo-cleavable morphology markers, genetically encoded tags, magnetic resonance image (MRI) contrast agents, or nucleic acid probes. In some embodiments, the images are microscope-derived images, comprising images from optical microscopes, electron microscopes, or scanning probe microscopes. In some embodiments, the images can comprise images from spatial molecular imagers. In some embodiments, the images are applied to a bleaching correction. In some embodiments, the transcriptomic assays comprise gene expression assays with fluorescently labeled probes, RNA sequencing (RNA-seq), microarray analysis, reverse transcription polymerase chain reaction (RT-PCR), cap analysis of gene expression, or single-cell RNA sequencing (scRNA-seq). In some embodiments, the software module configured to retrieve at least 1 image, at least 3 images, at least 5 images, at least 10 images, at least 15 images, at least 20 images, at least 30 images, at least 35 images, at least 40 images, at least 45 images, at least 50 images, at least 55 images, at least 60 images, at least 65 images, at least 70 images, at least 80 images, at least 90 images, at least 100 images, at least 120 images, at least 150 images, at least 200 images or more of the biological sample. In some embodiments, the biological sample is obtained at least in part of one or more of biopsy collection, surgical resection, xenograft, animal model, fine needle aspiration, peripheral blood collection, bone marrow biopsy, healthy tissue sampling, neoplastic tissue sampling, malignant tissue sampling, diseased tissue sampling, and implanted tissue sampling. In some embodiments, the biological sample comprises cells or tissues. In some embodiments, the cells comprise primary cells, stem cells, immune cells, carcinoma cells, sarcoma cells, lymphoma cells, melanoma cells, cancer cells, or neoplastic cells. In some embodiments, subcellular segmentation comprises nucleic segmentation, cytoplasm segmentation, protrusion segmentation or extra-cellular segmentation. In some embodiments, subcellular segmentation comprises training a machine learning algorithm and/or applying a machine learning algorithm.

In another aspect, disclosed herein are computer-implemented methods comprising: (a) retrieving, by a computer, a 3D scan image of a biological sample in High Dynamic Range (HDR) mode, wherein the biological sample is labeled with one of a plurality of morphology markers; (b.1) optionally a software module configured to reduce the 3D scan image to a 2D image, retaining the optimal focus regions from various z slices, using techniques such as maximum intensity projection, focus stacking and extended depth of field; (b.2) alternatively a software module configured to deconvolve the 2D images to improve the sharpness and clarity of the images while maintaining the z position of those images in their 3D scan image stack (c) retrieving, by the computer, a readout density map from transcriptomic assays of the biological sample; and (d) performing image preprocessing and segmentation on the biological sample based on the plurality of morphology markers or the readout density map. In some embodiments, the morphology markers comprise fluorescent dyes, nuclear stains, fluorescently labeled antibodies, immunohistochemistry (IHC) stains, photo-cleavable morphology markers, genetically encoded tags, magnetic resonance image (MRI) contrast agents, or nucleic acid probes.

In some embodiments, the images are microscope-derived images, comprising images from optical microscopes, electron microscopes, or scanning probe microscopes. In some embodiments, the images can comprise images from spatial molecular imagers. In some embodiments, the images are applied to a bleaching correction. In some embodiments, the transcriptomic assays comprise gene expression assays with fluorescently labeled probes, RNA sequencing (RNA-seq), microarray analysis, reverse transcription polymerase chain reaction (RT-PCR), cap analysis of gene expression, or single-cell RNA sequencing (scRNA-seq). In some embodiments, the software module is configured to retrieve at least 1 image, at least 3 images, at least 5 images, at least 10 images, at least 15 images, at least 20 images, at least 30 images, at least 35 images, at least 40 images, at least 45 images, at least 50 images, at least 55 images, at least 60 images, at least 65 images, at least 70 images, at least 80 images, at least 90 images, at least 100 images, at least 120 images, at least 150 images, at least 200 images or more of the biological sample. In some embodiments, the biological sample is obtained at least in part of one or more of biopsy collection, surgical resection, xenograft, animal model, fine needle aspiration, peripheral blood collection, bone marrow biopsy, healthy tissue sampling, neoplastic tissue sampling, malignant tissue sampling, diseased tissue sampling, and implanted tissue sampling. In some embodiments, the biological sample comprises cells or tissues. In some embodiments, the cells comprise primary cells, stem cells, immune cells, carcinoma cells, sarcoma cells, lymphoma cells, melanoma cells, cancer cells, or neoplastic cells. In some embodiments, subcellular segmentation comprises nucleic segmentation, cytoplasm segmentation, or extra-cellular segmentation. In some embodiments, subcellular segmentation comprises training a machine learning algorithm and/or applying a machine learning algorithm.

The systems, media, and methods disclosed herein comprise combining morphology staining with both the proteomic and transcriptomic data to bring an abundant information regarding cell structures including nuclei, cytoplasm and membrane regions. The systems, media, and methods disclosed herein comprise combining protein images expressing similar structures and/or cell types into multi-channel input morphology images. The systems, media, and methods disclosed herein comprise segmentation of extra-cellular processes. Various properties and statistics can be computed on per cell bases to enable researchers to study specific structures or regions of interest. Those properties and statistics allow the establishment of downstream analysis that use cell locations and per-cell fluorescence and shape properties as generated during segmentation. It also serves as a baseline performance across multiple sample types and serves as metrics for ground-truth-free segmentation evaluation of new datasets. In some embodiments, the evaluation of cell segmentation performance is based on direct comparison. In some embodiments, the evaluation of cell segmentation performance is achieved by projecting the statistics of new datasets to the embedding space created by the statistics of reference datasets of similar sample types.

In some embodiments, image preprocessing is performed on raw images before feeding into at least one cell segmentation module. In some embodiments, image preprocessing is based on conventional image processing techniques such as denoising, deblurring and image enhancements. In some embodiments, various denoising algorithms can be performed, including filter based (Wiener, Gaussian, Median), using wavelet transforms, total variation (TV) denoising, and deep learning-based approaches. In some embodiments, various deblurring algorithms can be performed, including the nearest-neighbor deconvolution, Richardson-Lucy deconvolution and total variation (TV) regularization. In some embodiments, various image enhancement algorithms can be performed, including histogram equalization and contrast limited adaptive histogram equalization (CLAHE), contrast stretching, gamma correction and unsharp masking.

In some embodiments, cell segmentation is based on algorithm comprising Cellpose Deep Learning algorithm, adaptive thresholding, Watershed algorithm, thresholding, region-based segmentation, active contours, Convolutional Neural Networks (CNN), Level Set Methods, Graph-based algorithms, fuzzy c-means clustering, Deep watershed, image morphology, or Markov Random Fields. In some embodiments, nuclei segmentation, cytoplasm segmentation, protrusion segmentation and extra-cellular segmentation are based on the same algorithm. In some embodiments, nuclei segmentation, cytoplasm segmentation, protrusion segmentation and extra-cellular segmentation are based on different algorithms.

In various embodiments, image segmentation software based on machine learning (ML) algorithms may be applied to create cell boundaries from fluorescent images of protein assays. In some embodiments, the protein assays may comprise protein antibodies binding to membrane proteins. In some embodiments, ML algorithms applied to image segmentation may comprise semantic segmentation, instance segmentation, or generative networks for segmentation. In some embodiments, image segmentation software may comprise ImageJ, CellProfiller, Cellpose, Ilastik, or QuPath, or any combination thereof.

In various embodiments, applications and use cases include, by way of non-limiting examples, discovering and mapping cell types and cell states, phenotypes of tissue microenvironment, differential expression of cell type based on spatial context, quantifying subcellular expression, and spatially resolved biomarker identification.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A better understanding of the features and advantages of the present subject matter will be obtained by reference to the following detailed description that sets forth illustrative embodiments and the accompanying drawings of which:

FIG. 1 shows a non-limiting example of a computing device; in this case, a device with one or more processors, memory, storage, and a network interface;

FIG. 2 shows a non-limiting example of a web/mobile application provision system; in this case, a system providing browser-based and/or native mobile user interfaces;

FIG. 3 shows a non-limiting example of a cloud-based web/mobile application provision system; in this case, a system comprising an elastically load balanced, auto-scaling web server and application server resources as well as synchronously replicated databases;

FIGS. 4A-4C show a non-limiting example of Multi-Modal Cell Acquisition and Segmentation System, FIG. 4A illustrates exemplary protein acquisition and 3D morphology sample collection, FIG. 4B illustrates exemplary RNA acquisition and detection, and FIG. 4C illustrates exemplary Sub-Cellular cell segmentation;

FIG. 5 shows a non-limiting example of High Dynamic Range (HDR) imaging method;

FIG. 6 shows a non-limiting example of two-step morphology scanning;

FIG. 7 shows a non-limiting example of an image affected by photo-bleaching from neighboring exposures of light;

FIGS. 8A-8C show a non-limiting example of scaling and normalization;

FIG. 9 shows a non-limiting example of Chromatic Aberration;

FIG. 10 shows a non-limiting example of Chromatic Corrections;

FIG. 11 shows a non-limiting example of spot density generation;

FIG. 12 shows a non-limiting example of generating density using grid;

FIGS. 13A and 13B show a non-limiting example of segmentation results based on morphology-stained and spot readout density, FIG. 13A shows non-limiting examples of several protein channels, FIG. 13B shows non-limiting examples of segmentation processed channels;

FIG. 14 shows a non-limiting example of diagram of Multi-Modal Cell Segmentation;

FIG. 15 shows a non-limiting example of processing cell segmentation using protein and spot density inputs;

FIG. 16 shows a non-limiting example of generation of new N-channel model;

FIGS. 17A and 17B show a non-limiting example of comparison of 2-Channel and 5-Channel model outputs, FIG. 17A shows a non-limiting example of various 2-Channel model outputs, FIG. 17B shows a non-limiting example of various 5-Channel model outputs;

FIGS. 18A-18D show a non-limiting example of dense neuron cells in human brain, FIG. 18A shows neuron cells with nuclei markers, FIG. 18B shows neuron cells with membrane markers, FIG. 18C shows astrocytes with GFAP markers, and FIG. 18D shows nuclei with DAPI markers;

FIG. 19 shows a non-limiting example of extra-cellular process segmentation;

FIGS. 20A-20C show a non-limiting example of results from cell segmentation, FIG. 20A shows a non-limiting example of a segmentation results overlay of a cell and extracellular processes in a human brain, FIG. 20B shows a non-limiting example of corresponding cell segmentation masks, FIG. 20C shows a non-limiting example of detection of extracellular cell segmentation masks;

FIG. 21 shows a non-limiting example of cell segmentation output providing sub-cellular and extra-cellular masks and statistics;

FIG. 22 illustrates a non-limiting example of an objective lens cone angle for a blurring function;

FIG. 23 illustrates a non-limiting example of a 3D segmentation pipeline;

FIG. 24 illustrates image preprocessing techniques such as image sharpening and enhancement;

FIG. 25 illustrates a non-limiting example of an image acquired using High Definition Range (HDR) settings;

FIG. 26 illustrates a non-limiting example of output produced by the nearest neighbor (NN) deconvolution method;

FIG. 27 illustrates a non-limiting example of cell protrusion merging;

FIG. 28 illustrates a non-limiting example of Intersection Of Union (IoU) merging results between a nuclei segmentation output and a membrane plus nuclei segmentation;

FIGS. 29A-29C illustrate a non-limiting example of intersection analysis, FIG. 29A illustrates a non-limiting example of IoU merging results in 2D image slices, FIG. 29B illustrates a non-limiting example of IoU merging results within a 3D volume, FIG. 29C shows a non-limiting example of one cell marked in all z-slices throughout the entire Z-stack; and

FIG. 30 illustrates a non-limiting example of segmentation of cells of various tissues.

### DETAILED DESCRIPTION

Described herein, in certain embodiments, are systems comprising at least one processor and instructions executable by the at least one processor to provide a multi-modal segmentation application comprising: (a) a software module configured to retrieve a 3D scan image of a biological sample in High Dynamic Range (HDR) mode, wherein the biological sample is labeled with one of a plurality of morphology markers; (b) a software module configured to reduce the 3D scan image to a 2D image and obtain an optimal focus region in a z slice, or utilize the entire 3D stack where each 2D z-slice has been deconvolved to enhance features and reduce blur; (c) a software module configured to retrieve a readout density map from transcriptomic assays of the biological sample; and (d) a software module configured to perform subcellular segmentation on the biological sample based on the plurality of morphology markers or the readout density map. In some embodiments, the morphology markers can comprise one or more fluorescent dyes, nuclear stains, fluorescently labeled antibodies, immunohistochemistry (IHC) stains, photo-cleavable morphology markers, genetically encoded tags, magnetic resonance image (MRI) contrast agents, or nucleic acid probes, or any combination thereof.

In some embodiments, the images can comprise microscope-derived images. In some embodiments, the microscope-derived images can comprise images from optical microscopes, electron microscopes, or scanning probe microscopes. In some embodiments, the images can comprise images from spatial molecular imagers. In some embodiments, a bleaching correction can be applied to the images. In some embodiments, the transcriptomic assays can comprise one or more of gene expression assays with fluorescently labeled probes, RNA sequencing (RNA-seq), microarray analysis, reverse transcription polymerase chain reaction (RT-PCR), cap analysis of gene expression, or single-cell RNA sequencing (scRNA-seq), or any combination thereof. In some embodiments, the software module can be configured to retrieve at least 1 image, at least 3 images, at least 5 images, at least 10 images, at least 15 images, at least 20 images, at least 30 images, at least 35 images, at least 40 images, at least 45 images, at least 50 images, at least 55 images, at least 60 images, at least 65 images, at least 70 images, at least 80 images, at least 90 images, at least 100 images, at least 120 images, at least 150 images, at least 200 images, or more than at least 200 images of the biological sample. In some embodiments, the biological sample can be obtained at least in part of from one or more of biopsy collection, surgical resection, xenograft, animal model, fine needle aspiration, peripheral blood collection, bone marrow biopsy, healthy tissue sampling, neoplastic tissue sampling, malignant tissue sampling, diseased tissue sampling, or implanted tissue sampling, or any combination thereof. In some embodiments, the biological sample can comprise cells or tissues. In some embodiments, the cells can comprise one or more of primary cells, stem cells, immune cells, carcinoma cells, sarcoma cells, lymphoma cells, melanoma cells, cancer cells, or neoplastic cells, or any combination thereof. In some embodiments, subcellular segmentation can comprise one or more of nucleic segmentation, cytoplasm segmentation, or extra-cellular segmentation, or any combination thereof. In some embodiments, subcellular segmentation can comprise training a machine learning algorithm, or applying a machine learning algorithm, or both.

Also described herein, in certain embodiments, are non-transitory computer-readable storage media encoded with instructions executable by one or more processors that can provide a multi-modal segmentation application comprising: (a) a software module configured to retrieve a 3D scan image of a biological sample in High Dynamic Range (HDR) mode, wherein the biological sample is labeled with one of a plurality of morphology markers; (b.1) optionally a software module configured to reduce the 3D scan image to a 2D image, retaining the optimal focus regions from various z slices, using techniques such as maximum intensity projection, focus stacking and extended depth of field; (b.2) alternatively a software module configured to deconvolve the 2D images to improve the sharpness and clarity of the images while maintaining the z position of those images in their 3D scan image stack (c) a software module configured to retrieve a readout density map from transcriptomic assays of the biological sample; and (d) a software module configured to perform subcellular segmentation on the biological sample based on the plurality of morphology markers or the readout density map. In some embodiments, the morphology markers can comprise one or more of fluorescent dyes, nuclear stains, fluorescently labeled antibodies, immunohistochemistry (IHC) stains, photo-cleavable morphology markers, genetically encoded tags, magnetic resonance image (MRI) contrast agents, or nucleic acid probes, or any combination thereof. In some embodiments, the images can be microscope-derived images. In some embodiments, microscope-derived images can comprise images from optical microscopes, electron microscopes, or scanning probe microscopes, or any combination thereof. In some embodiments, the images can comprise images from spatial molecular imagers. In some embodiments, a bleaching correction can be applied to the images. In some embodiments, the transcriptomic assays can comprise one or more of gene expression assays with fluorescently labeled probes, RNA sequencing (RNA-seq), microarray analysis, reverse transcription polymerase chain reaction (RT-PCR), cap analysis of gene expression, or single-cell RNA sequencing (scRNA-seq), or any combination thereof. In some embodiments, the software module can be configured to retrieve at least 1 image, at least 3 images, at least 5 images, at least 10 images, at least 15 images, at least 20 images, at least 30 images, at least 35 images, at least 40 images, at least 45 images, at least 50 images, at least 55 images, at least 60 images, at least 65 images, at least 70 images, at least 80 images, at least 90 images, at least 100 images, at least 120 images, at least 150 images, at least 200 images, or more than at least 200 images of the biological sample. In some embodiments, the biological sample can be obtained at least in part from one or more of biopsy collection, surgical resection, xenograft, animal model, fine needle aspiration, peripheral blood collection, bone marrow biopsy, healthy tissue sampling, neoplastic tissue sampling, malignant tissue sampling, diseased tissue sampling, and implanted tissue sampling, or any combination thereof. In some embodiments, the biological sample can comprise cells or tissues. In some embodiments, the cells can comprise primary cells, stem cells, immune cells, carcinoma cells, sarcoma cells, lymphoma cells, melanoma cells, cancer cells, or neoplastic cells, or any combination thereof. In some embodiments, subcellular segmentation can comprise nucleic segmentation, cytoplasm segmentation, or extra-cellular segmentation, or any combination thereof. In some embodiments, subcellular segmentation can comprise training a machine learning algorithm, applying a machine learning algorithm, or both.

In another aspect also described herein are computer-implemented methods which can comprise: (a) retrieving, by a computer, a 3D scan image of a biological sample in High Dynamic Range (HDR) mode, wherein the biological sample can be labeled with one of a plurality of morphology markers (b.1) optionally a software module configured to reduce the 3D scan image to a 2D image, retaining the optimal focus regions from various z slices, using techniques such as maximum intensity projection, focus stacking and extended depth of field; (b.2) alternatively a software module which can be configured to deconvolve the 2D images to improve the sharpness and clarity of the images while maintaining the z position of those images in their 3D scan image stack (c) retrieving, by the computer, a readout density map from transcriptomic assays of the biological sample; and (d) performing segmentation on the biological sample based on the plurality of morphology markers or the readout density map. In some embodiments, the morphology markers can comprise one or more of fluorescent dyes, nuclear stains, fluorescently labeled antibodies, immunohistochemistry (IHC) stains, photo-cleavable morphology markers, genetically encoded tags, magnetic resonance image (MRI) contrast agents, or nucleic acid probes, or any combination thereof. In some embodiments, the images can be microscope-derived images. In some embodiments, the microscope-derived images can comprise images from optical microscopes, electron microscopes, or scanning probe microscopes, or any combination thereof. In some embodiments, the images can comprise images from spatial molecular imagers. In some embodiments, a bleaching correction can be applied to the images. In some embodiments, the transcriptomic assays can comprise gene expression assays comprising one or more of fluorescently labeled probes, RNA sequencing (RNA-seq), microarray analysis, reverse transcription polymerase chain reaction (RT-PCR), cap analysis of gene expression, or single-cell RNA sequencing (scRNA-seq), or any combination thereof. In some embodiments, the software module can be configured to retrieve at least 1 image, at least 3 images, at least 5 images, at least 10 images, at least 15 images, at least 20 images, at least 30 images, at least 35 images, at least 40 images, at least 45 images, at least 50 images, at least 55 images, at least 60 images, at least 65 images, at least 70 images, at least 80 images, at least 90 images, at least 100 images, at least 120 images, at least 150 images, at least 200 images, or more than at least 200 images of the biological sample. In some embodiments, the biological sample can be obtained at least in part from one or more of biopsy collection, surgical resection, xenograft, animal model, fine needle aspiration, peripheral blood collection, bone marrow biopsy, healthy tissue sampling, neoplastic tissue sampling, malignant tissue sampling, diseased tissue sampling, and implanted tissue sampling, or any combination thereof. In some embodiments, the biological sample can comprise cells or tissues. In some embodiments, the cells can comprise one or more of primary cells, stem cells, immune cells, carcinoma cells, sarcoma cells, lymphoma cells, melanoma cells, cancer cells, or neoplastic cells, or any combination thereof. In some embodiments, subcellular segmentation can comprise one or more of nucleic segmentation, cytoplasm segmentation, or extra-cellular segmentation, or any combination thereof. In some embodiments, subcellular segmentation can comprise training a machine learning algorithm, applying a machine learning algorithm, or both.

### DEFINITIONS

Unless otherwise defined, all technical terms used herein have the same meaning as commonly understood by one of ordinary skill in the art to which the present subject matter belongs.

As used in this specification and the appended claims, the singular forms “a,” “an,” and “the” include plural references unless the context clearly dictates otherwise. Any reference to “or” herein is intended to encompass “and/or” unless otherwise stated.

Reference throughout this specification to “some embodiments,” “further embodiments,” or “a particular embodiment,” means that a particular feature, structure, or characteristic described in connection with the embodiment is included in at least one embodiment. Thus, the appearances of the phrase “in some embodiments,” or “in further embodiments,” or “in a particular embodiment” in various places throughout this specification are not necessarily all referring to the same embodiment. Furthermore, the particular features, structures, or characteristics may be combined in any suitable manner in one or more embodiments.

### Multi-Modal Cell Acquisition and Segmentation System

In some embodiments, the systems, media, and methods disclosed herein can comprise combining morphology staining with both the proteomic and transcriptomic data. In some embodiments, the systems, media, and methods disclosed herein can further comprise receiving information regarding cell structures. In some embodiments, cell structures can include nuclei, cytoplasm, or membrane regions, or any combination thereof. In some embodiments, the systems, media, and methods disclosed herein can comprise combining protein images expressing similar structures into multi-channel input morphology images. In some embodiments, the systems, media, and methods disclosed herein can comprise combining protein images expressing similar cell types into multi-channel input morphology images. In some embodiments, the systems, media, and methods disclosed herein can comprise combining protein images expressing similar cell types and similar structures into multi-channel input morphology images. In some embodiments, the multi-channel input images can comprise at least 1-channel input morphology images, at least 3-channel input morphology images, at least 5-channel input morphology images, at least 10-channel input morphology images, at least 15-channel input morphology images, at least 20-channel input morphology images, at least 30-channel input morphology images, at least 35-channel input morphology images, at least 40-channel input morphology images, at least 45-channel input morphology images, at least 50-channel input morphology images, at least 55-channel input morphology images, at least 60-channel input morphology images, at least 65-channel input morphology images, at least 70-channel input morphology images, at least 80-channel input morphology images, at least 90-channel input morphology images, at least 100-channel input morphology images, at least 120-channel input morphology images, at least 150-channel input morphology images, or at least 200-channel input morphology images, or more than at least 200-channel input morphology images, including increments therein.

In some embodiments, the systems, media, and methods disclosed herein can comprise a multi-step process of segmentation. In some embodiments, the multi-step process of segmentation can comprise segmenting regions of nuclei. In some embodiments, the multi-step process of segmentation can comprise segmenting regions of cytoplasm. In some embodiments, the multi-step process of segmentation can comprise segmenting regions of membrane. In some embodiments, the systems, media, and methods disclosed herein can comprise segmenting of extra-cellular objects to segment regions. In some embodiments, the segment regions can be segment regions of an organ or tissue. In some embodiments, the segment regions can be segment regions of the brain. In some embodiments, the segment regions can be distal or disconnected to the soma, or both. In some embodiments, the systems, media, and methods disclosed herein can comprise combining images expressing similar structures into one or more input morphology images. In some embodiments, the systems, media, and methods disclosed herein can comprise combining images expressing similar cell types into one or more input morphology images. In some embodiments, the systems, media, and methods disclosed herein can comprise combining images expressing similar structures into one or more input morphology images and cell types into one or more input morphology images. In some embodiments, the input morphology images can be multi-channel input morphology images. In some embodiments, the systems, media, and methods disclosed herein can comprise combining images expressing similar structures into 5-channel input morphology images. In some embodiments, the systems, media, and methods disclosed herein can comprise combining images expressing similar cell types into 5-channel input morphology images. In some embodiments, the systems, media, and methods disclosed herein can comprise combining images expressing similar structures into 5-channel input morphology images and cell types into 5-channel input morphology images.

In some embodiments, the systems, media, and methods disclosed herein can comprise combining one or more protein images. In some embodiments, the number of protein images combined can be about 1, about 2, about 3, about 4, about 5, about 10, about 20, about 30, about 40, about 50, about 60, about 70, about 80, about 90, about 100, about 120, about 140, about 160, about 180, about 200, or more than, a about 200 protein images. In some embodiments, the systems, media, and methods disclosed herein can comprise processing an additional input channel of raw RNA spot readout density to supplement missing areas of signal from the morphology markers.

In some embodiments, the systems, media, and methods disclosed herein can comprise segmentation of extra-cellular processes. In some embodiments, the systems, media, and methods disclosed herein can further comprise labeling one or more images with a unique id for each cell. In some embodiments, the systems, media, and methods disclosed herein can further comprise labeling one or more images with a unique id for each one of the connected processes. In some embodiments, the systems, media, and methods disclosed herein can further comprise labeling one or more images with a unique id for each cell and each one of the connected processes. The systems, media, and methods disclosed herein can further comprise identifying a “compartment label” for a different compartment of the area for each cell.

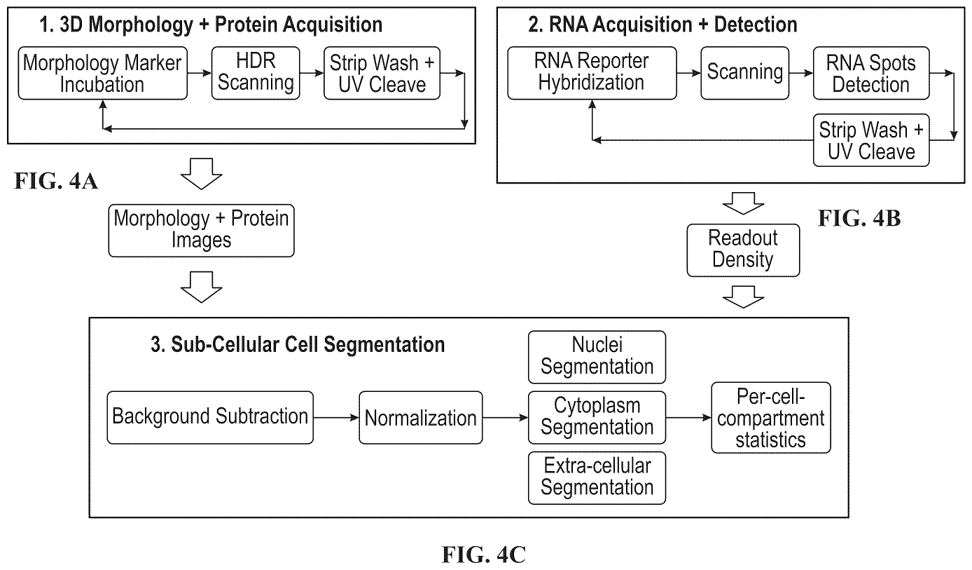

As a non-limiting example, a multi-modal cell acquisition and segmentation system is illustrated in FIGS. 4A-4C. For example, protein acquisition and 3D morphology samples were collected as illustrated in FIG. 4A. Protein samples and a morphology marker, for example, a photo-cleavable marker, were incubated and scanned into 3D images in High Dynamic Range (HDR) mode. Next, for example, markers were cleared using for example UV illumination and chemical wash in the tissue. For example, repeat re-staining and clearing of markers using UV illumination and chemical wash in the tissue were performed. As an illustrative example, RNA acquisition and detection were performed as illustrated in FIG. 4B. Next, for example, after scanning RNA reporter hybridization samples, RNA spots were detected. Next, for example, markers were cleared using UV illumination and wash in the samples.

In some embodiments, the systems, media, and methods disclosed herein can comprise retrieving images from one or more tissue scans. In some embodiments, the one or more tissue scans can comprise one or more scans of one or more tissues stained with morphology markers. In some embodiments, one or more tissue scans can comprise one or more scans of one or more tissues incubated with protein reporters. In some embodiments, one or more tissue scans can comprise one or more scans of one or more tissues incubated with RNA reporters. In some embodiments, the systems, media, and methods disclosed herein can comprise removing one or more reporters from the one or more tissue samples. In some embodiments, one or more reporters can be reduced in one or more tissue samples. In some embodiments, one or more reporters can be bleached, cleaved, or both in one or more tissue samples. In some embodiments, reporters can be reduced in one or more tissue samples by applying one or more of UV light, heat, proteases, endonuclease, nucleases, esterases, ribonucleases, RNase A, RNase TI, RNase H, disulfate bond reducing agents (e.g., dithiothreitol, tris(2-carboxyethyl) phosphine), salt buffer, alkali, hydrogen bond destabilization solvents (e.g., formamide, DMSO), or any combination thereof.

In some non-limiting examples, after processing images of one or more proteins using morphology and density measurements of RNA detection, Sub-Cellular cell segmentation was performed as shown in, for example, FIG. 4C. As illustrated in FIG. 4C, cell segmentation processes included performing, for example, background subtraction, normalization, segmentation for regions to nuclei, regions to cytoplasm or regions to membrane, or any combination thereof. In some embodiments, different compartments of the area of each cell can be identified by a unique “compartment label.” In some embodiments, various properties and statistics can be computed for each cell. In some embodiments, various properties and statistics computed for each cell can provide information concerning specific structures or regions of interest, or both.

In some embodiments, the systems, media, and methods disclosed herein can comprise retrieving images of biological samples. In some embodiments, the retrieved images of biological examples can be labeled with morphology markers. In some embodiments, the morphology markers can comprise one or more of fluorescent dyes, nuclear stains, fluorescently labeled antibodies, immunohistochemistry (IHC) stains, photo-cleavable morphology markers, genetically encoded tags, magnetic resonance image (MRI) contrast agents, or nucleic acid probes, or any combination thereof.

In some embodiments, the systems, media, and methods disclosed herein can comprise generating a readout density map using transcriptomic assays of biological samples. In some embodiments, the density map can be a heatmap, a choropleth map, a kernel density map, a dot density map, a contour map, or a proportional symbol map. In some cases, the heatmap can further comprise a graphical representation of data where the values are depicted using colors. In some cases, the heatmap can be used to visualize the density or intensity of a particular phenomenon across a two-dimensional space. In some cases, the two-dimensional space can be geographical areas, images, or grids, or any combination thereof. In some embodiments, the density map can show the density or concentration of a particular attribute or event within a given area. In some embodiments, the density map can provide visual information about the distribution and intensity of data points or events across a spatial domain.

In some embodiments, the transcriptomic assays can comprise one or more of gene expression assays with fluorescently labeled probes, RNA sequencing (RNA-seq), microarray analysis, reverse transcription polymerase chain reaction (RT-PCR), cap analysis of gene expression, or single-cell RNA sequencing (scRNA-seq), or any combination thereof.

In some embodiments, the systems, media, and methods disclosed herein can comprise retrieving images of biological samples. In some embodiments, the biological samples can be obtained at least in part using one or more of biopsy collection, surgical resection, xenograft, animal model, fine needle aspiration, peripheral blood collection, bone marrow biopsy, healthy tissue sampling, neoplastic tissue sampling, malignant tissue sampling, diseased tissue sampling, or implanted tissue sampling, or any combination thereof.

In some embodiments, the biological samples can comprise cells or tissues, or both. In some embodiments, the cells can comprise one or more of primary cells, stem cells, immune cells, carcinoma cells, sarcoma cells, lymphoma cells, melanoma cells, cancer cells, or neoplastic cells, or any combination thereof.

In some embodiments, the systems, media, and methods disclosed herein can comprise retrieving images of spatially resolved high-plex gene expression data from tissue. In some embodiments, the systems, media, and methods disclosed herein can comprise retrieving images of spatially resolved high-plex protein data from tissue. In some embodiments, the systems, media, and methods disclosed herein can comprise retrieving images of RNA assays. In some embodiments, the systems, media, and methods disclosed herein can comprise retrieving images of RNA assays to profile an entire transcriptome. In some embodiments, the systems, media, and methods disclosed herein can comprise retrieving images of RNA assays to profile an entire transcriptome from tissues on a single formalin-fixed paraffin-embedded (FFPE) sample. In some embodiments, the systems, media, and methods disclosed herein can comprise retrieving images of RNA assays to profile an entire transcriptome from tissues on a fresh frozen (FF) sample slide. In some embodiments, the systems, media, and methods disclosed herein can comprise retrieving images of protein assays to generate quantitative analysis, spatial analysis, or both, of multiple proteins. In some embodiments, the quantitative analysis, spatial analysis, or both, of multiple proteins can be generated from a single FFPE or FF sample slide. In some embodiments, FFPE or FF tissue sections may be stained with barcoded in-situ hybridization probes that bind to endogenous mRNA transcripts. In some embodiments, a user may select regions of the interest (ROI) to profile. In some embodiments, each ROI segment can be further sub-divided into areas of illumination (AOI) based on tissue morphology. In some embodiments, a spatial molecular imager may photo-cleave each AOI segment separately. In some embodiments, a spatial molecular imager may collect expression tags or barcodes for each AOI segment separately. In some embodiments, the tags or barcodes may be used for downstream sequencing or data processing, or both.

### Computing System

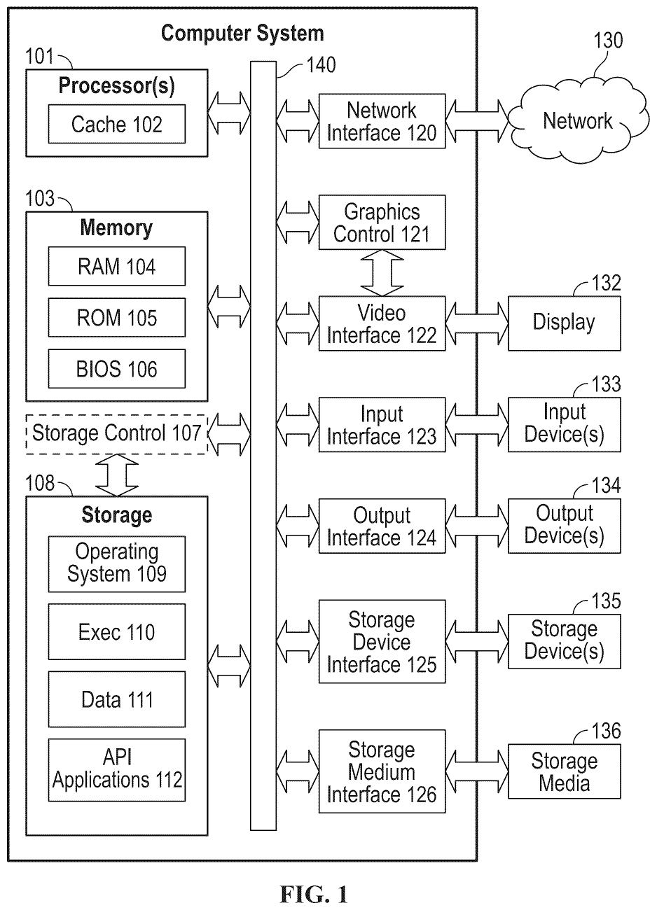

Referring to FIG. 1, a block diagram is shown depicting an exemplary machine that includes a computer system 100 (e.g., a processing or computing system) within which a set of instructions can execute for causing a device to perform or execute any one or more of the aspects and/or methodologies for static code scheduling of the present disclosure. The components herein are examples only and do not limit the scope of use or functionality of any hardware, software, embedded logic component, or a combination of two or more such components implementing particular embodiments.

Computer system 100 may include one or more processors 101, a memory 103, and a storage 108 that communicate with each other, and with other components, via a bus 140. The bus 140 may also link a display 132, one or more input devices 133 (which may, for example, include a keypad, a keyboard, a mouse, a stylus, etc.), one or more output devices 134, one or more storage devices 135, and various tangible storage media 136. All of these elements may interface directly or via one or more interfaces or adaptors to the bus 140. For instance, the various tangible storage media 136 can interface with the bus 140 via storage medium interface 126. Computer system 100 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 100 includes one or more processor(s) 101 (e.g., central processing units (CPUs), general purpose graphics processing units (GPGPUs), or quantum processing units (QPUs)) that carry out functions. Processor(s) 101 optionally contains a cache memory unit 102 for temporary local storage of instructions, data, or computer addresses. Processor(s) 101 are configured to assist in execution of computer readable instructions. Computer system 100 may provide functionality for the components depicted in FIG. 1 as a result of the processor(s) 101 executing non-transitory, processor-executable instructions embodied in one or more tangible computer-readable storage media, such as memory 103, storage 108, storage devices 135, and/or storage medium 136. The computer-readable media may store software that implements particular embodiments, and processor(s) 101 may execute the software. Memory 103 may read the software from one or more other computer-readable media (such as mass storage device(s) 135, 136) or from one or more other sources through a suitable interface, such as network interface 120. The software may cause processor(s) 101 to carry out one or more processes or one or more steps of one or more processes described or illustrated herein. Carrying out such processes or steps may include defining data structures stored in memory 103 and modifying the data structures as directed by the software.

The memory 103 may include various components (e.g., machine readable media) including, but not limited to, a random access memory component (e.g., RAM 104) (e.g., static RAM (SRAM), dynamic RAM (DRAM), ferroelectric random access memory (FRAM), phase-change random access memory (PRAM), etc.), a read-only memory component (e.g., ROM 105), and any combinations thereof. ROM 105 may act to communicate data and instructions unidirectionally to processor(s) 101, and RAM 104 may act to communicate data and instructions bidirectionally with processor(s) 101. ROM 105 and RAM 104 may include any suitable tangible computer-readable media described below. In one example, a basic input/output system 106 (BIOS), including basic routines that help to transfer information between elements within computer system 100, such as during start-up, may be stored in the memory 103.

Fixed storage 108 is connected bidirectionally to processor(s) 101, optionally through storage control unit 107. Fixed storage 108 provides additional data storage capacity and may also include any suitable tangible computer-readable media described herein. Storage 108 may be used to store operating system 109, executable(s) 110, data 111, applications 112 (application programs), and the like. Storage 108 can also include an optical disk drive, a solid-state memory device (e.g., flash-based systems), or a combination of any of the above. Information in storage 108 may, in appropriate cases, be incorporated as virtual memory in memory 103.

In one example, storage device(s) 135 may be removably interfaced with computer system 100 (e.g., via an external port connector (not shown)) via a storage device interface 125. Particularly, storage device(s) 135 and an associated machine-readable medium may provide non-volatile and/or volatile storage of machine-readable instructions, data structures, program modules, and/or other data for the computer system 100. In one example, software may reside, completely or partially, within a machine-readable medium on storage device(s) 135. In another example, software may reside, completely or partially, within processor(s) 101.

Bus 140 connects a wide variety of subsystems. Herein, reference to a bus may encompass one or more digital signal lines serving a common function, where appropriate. Bus 140 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 100 may also include an input device 133. In one example, a user of computer system 100 may enter commands and/or other information into computer system 100 via input device(s) 133. Examples of an input device(s) 133 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 133 may be interfaced to bus 140 via any of a variety of input interfaces 123 (e.g., input interface 123) including, but not limited to, serial, parallel, game port, USB, FIREWIRE, THUNDERBOLT, or any combination of the above.

In particular embodiments, when computer system 100 is connected to network 130, computer system 100 may communicate with other devices, specifically mobile devices and enterprise systems, distributed computing systems, cloud storage systems, cloud computing systems, and the like, connected to network 130. Communications to and from computer system 100 may be sent through network interface 120. For example, network interface 120 may receive incoming communications (such as requests or responses from other devices) in the form of one or more packets (such as Internet Protocol (IP) packets) from network 130, and computer system 100 may store the incoming communications in memory 103 for processing. Computer system 100 may similarly store outgoing communications (such as requests or responses to other devices) in the form of one or more packets in memory 103 and communicated to network 130 from network interface 120. Processor(s) 101 may access these communication packets stored in memory 103 for processing.

Examples of the network interface 120 include, but are not limited to, a network interface card, a modem, and any combination thereof. Examples of a network 130 or network segment 130 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 130, may employ a wired and/or a wireless mode of communication. In general, any network topology may be used.

Information and data can be displayed through a display 132. Examples of a display 132 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 132 can interface to the processor(s) 101, memory 103, and fixed storage 108, as well as other devices, such as input device(s) 133, via the bus 140. The display 132 is linked to the bus 140 via a video interface 122, and transport of data between the display 132 and the bus 140 can be controlled via the graphics control 121. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOYE VR, Zeiss VR One, Avegant Glyph, Freefly VR headset, and the like. In still further embodiments, the display is a combination of devices such as those disclosed herein.

In addition to a display 132, computer system 100 may include one or more other peripheral output devices 134 including, but not limited to, an audio speaker, a printer, a storage device, and any combinations thereof. Such peripheral output devices may be connected to the bus 140 via an output interface 124. Examples of an output interface 124 include, but are not limited to, a serial port, a parallel connection, a USB port, a FIREWIRE port, a THUNDERBOLT port, and any combinations thereof.

In addition or as an alternative, computer system 100 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, distributed computing systems, cloud computing platforms, server clusters, server computers, desktop computers, laptop computers, notebook computers, sub-notebook computers, netbook computers, netpad computers, handheld computers, Internet appliances, mobile smartphones, and tablet computers. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

### Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program can include a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, XML, and document-oriented database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application can be written to some extent in a presentation definition language such as Cascading Style Sheets (CSS). In some embodiments, a web application can be written to some extent in a client-side scripting language such as Asynchronous JavaScript and XML (AJAX), Flash® ActionScript, JavaScript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tel, Smalltalk, WebDNA®, or Groovy. In some embodiments, a web application can be written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

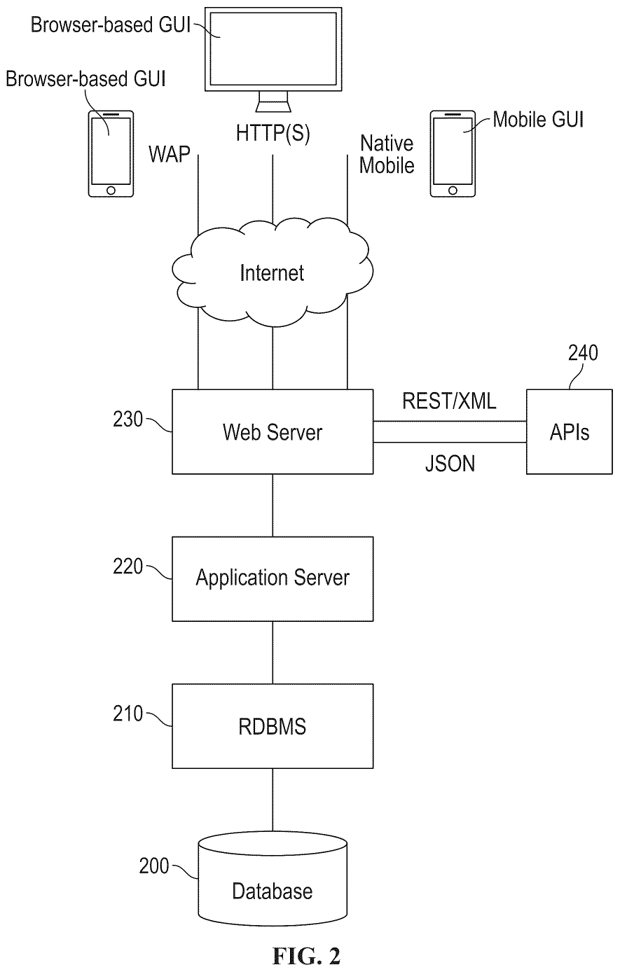

Referring to FIG. 2, in a particular embodiment, an application provision system comprises one or more databases 200 accessed by a relational database management system (RDBMS) 210. Suitable RDBMSs include Firebird, MySQL, PostgreSQL, SQLite, Oracle Database, Microsoft SQL Server, IBM DB2, IBM Informix, SAP Sybase, Teradata, and the like. In this embodiment, the application provision system further comprises one or more application servers 220 (such as Java servers, .NET servers, PHP servers, and the like) and one or more web servers 230 (such as Apache, IIS, GWS and the like). The web server(s) optionally expose one or more web services via app application programming interfaces (APIs) 240. Via a network, such as the Internet, the system provides browser-based and/or mobile native user interfaces.

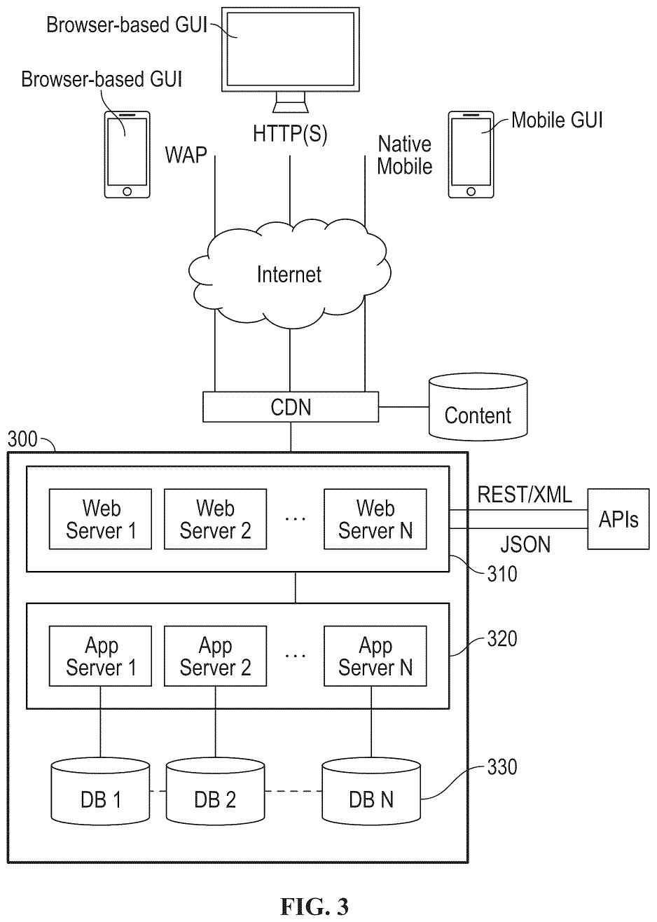

Referring to FIG. 3, in a particular embodiment, an application provision system alternatively has a distributed, cloud-based architecture 300 and comprises elastically load balanced, auto-scaling web server resources 310 and application server resources 320 as well synchronously replicated databases 330.

### Standalone Application

In some embodiments, a computer program can include a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB. NET, or combinations thereof. Compilation is often performed, at least in part, to create an executable program. In some embodiments, a computer program can include one or more executable complied applications.

### Software Modules

In some embodiments, the platforms, systems, media, and methods disclosed herein can include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module can comprise a file, a section of code, a programming object, a programming structure, a distributed computing resource, a cloud computing resource, or combinations thereof. In further various embodiments, a software module can comprise a plurality of files, a plurality of sections of code, a plurality of programming objects, a plurality of programming structures, a plurality of distributed computing resources, a plurality of cloud computing resources, or combinations thereof. In various embodiments, the one or more software modules comprise, by way of non-limiting examples, a web application, a mobile application, a standalone application, and a distributed or cloud computing application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

### Databases

In some embodiments, the systems, media, and methods disclosed herein can include one or more databases, or use of the same. In view of the disclosure provided herein, those of skill in the art will recognize that many databases are suitable for storage and retrieval of user information, study information, slide information, field of view (FoV) information, flow cell information, image information, genomic information, transcriptomic information, and proteomic information. In various embodiments, suitable databases include, by way of non-limiting examples, relational databases, non-relational databases, object-oriented databases, object databases, entity-relationship model databases, associative databases, XML databases, document-oriented databases, and graph databases. Further non-limiting examples include SQL, PostgreSQL, MySQL, Oracle, DB2, Sybase, and MongoDB. In some embodiments, a database can be Internet-based. In further embodiments, a database is web-based. In still further embodiments, a database can be cloud computing-based. In a particular embodiment, a database can be a distributed database. In other embodiments, a database can be based on one or more local computer storage devices.

In some embodiments, data stored on databases can include, biological image data. In some embodiments, biological image data can include, in some non-limiting examples, microscopy images (e.g., micrographs) of formalin fixed paraffin embedded (FFPE) samples of cells, microscopy images (e.g., micrographs) of formalin fixed paraffin embedded (FFPE) samples of tissues, microscopy images (e.g., micrographs) of fresh frozen (FF) samples of cells, or microscopy images (e.g., micrographs) of fresh frozen (FF) samples of tissues, or any combination thereof. In some embodiments, data from a single slide can be split into two datasets. In some cases, the two datasets can comprise an RNA Assays dataset and a Protein Assays dataset. In some embodiments, data from a single slide can be combined into one dataset. In some embodiments, the one dataset can include both RNA assays data and Protein Assays data. In some embodiments, the image data can be two-dimensional image data. In some embodiments, the image data can be three-dimensional image data. In some embodiments, the data includes, by way of non-limiting examples, “-omics” data such as genomic data, proteomic data, metabolomic data, metagenomic data, phenomic data, or transcriptomic data, or any combination thereof. In some embodiments, the -omics data can be associated with the image data. In some embodiments, the -omics data can be spatially associated with the image data. In some cases, the -omics data can be spatially associated with the image data in two dimensions, three-dimensions, or both. In some embodiments, the -omics data can be associated with the image data as metadata or as an overlay to the image, or both. In some embodiments, data can comprise, in some non-limiting examples, patient data, demographic data, diagnosis data, disease data, treatment data, study data, or any combination thereof.

### Image Acquisition

The systems, media, and methods disclosed herein can comprise retrieving images from one or more tissue scans of tissue stained with morphology markers. In some embodiments, the tissue can be incubated with protein reporters. In some embodiments, the tissue can be incubated with RNA reporters. In some embodiments, the systems, media, and methods disclosed herein can comprise removing reporters from the tissue. In some embodiments, reporters can be bleached. In some embodiments, reporters can be cleaved. In some embodiments, reporters can be bleached, cleaved, or both, by applying one or more of UV light, heat, proteases, endonuclease, nucleases, esterases, ribonucleases, RNase A, RNase Tl, RNase H, disulfate bond reducing agents (e.g., dithiothreitol, tris(2-carboxyethyl)phosphine), salt buffer, alkali, hydrogen bond destabilization solvents (e.g., formamide, DMSO) or any combination thereof.

In some embodiments, the systems, media, and methods disclosed herein can comprise retrieving images. In some embodiments, the images can comprise microscope-derived images. In some embodiments, the microscope-derived images can comprise images from optical microscopes, electron microscopes, or scanning probe microscopes, or any combination thereof. In some embodiments, the images can comprise images from spatial molecular imagers.

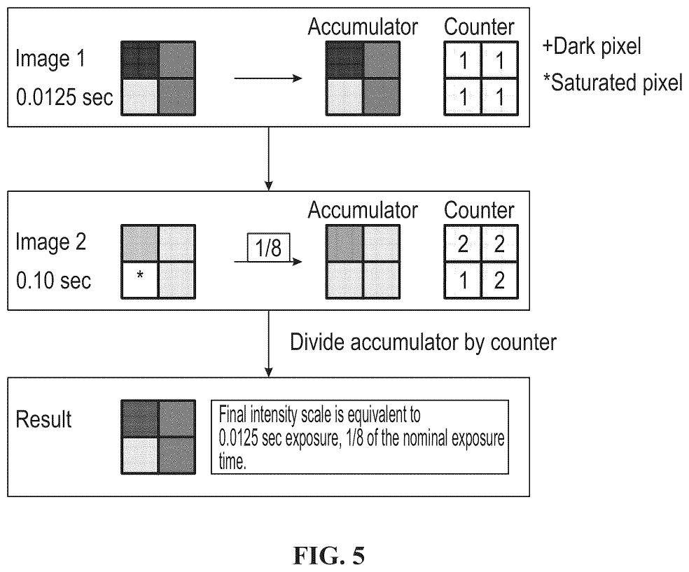

In some embodiments, morphology signals are acquired. In some embodiments, RNA protein signals are acquired. In some embodiments, both morphology signals and RNA protein signals are acquired. In some embodiments, the morphology and RNA protein signals are acquired using the high dynamic range imaging (HDR) mode. In some embodiments, the signals are acquired using HDR to improve the dynamic range of the signal. As illustrated in FIG. 5, Image I was, in one non-limiting example, obtained with exposure of 0.0125 second with details in the bright area, while image 2 was obtained with exposure of 0.1 second with details in the dark area. In some embodiments, the result image can be generated by dividing an accumulator by a counter. As illustrated in one non-limiting example of FIG. 5, the final intensity scale was equivalent to exposure with 0.0125 second, ⅛ of the nominal exposure time.

In some embodiments, the systems, media, and methods disclosed herein can comprise combining protein images expressing similar structures or cell types, or both, into multi-channel input morphology images. In some embodiments, the systems, media, and methods disclosed herein can comprise adding an additional input channel. In some embodiments, the additional input channel can comprise raw RNA spot density input to supplement missing signal from the morphology markers. In some embodiments, a spatial molecular imager can be used to image nuclei and DAPI for staining. In some cases, the spatial molecular imager can be a CosMx™ instrument. In some embodiments, the systems, media, and methods disclosed herein can comprise biological samples labeled with photocleavable morphology markers. In some embodiments, the DAPI channel acquisition for one FOV can cause cleaving or bleaching of reporters, or both, in adjacent FOVs. In some embodiments, order of image or channel acquisition, or both, is determined. In some cases, order of image or channel acquisition can be determined to prevent visible and unrecoverable signal loss along the edges of the FOVs, particularly in contiguous scans.

In some embodiments, the systems, media, and methods disclosed herein can comprise combining multi-channel input morphology images. In some embodiments, the morphology images can be used for different markers expressing nuclei, cytoplasm, or cell membrane, or any combination thereof. In one non-limiting example, the 5-channel morphology images used for segmentation can comprise Blue (B), Green (G), Yellow (Y), Red (R) and UV (U).

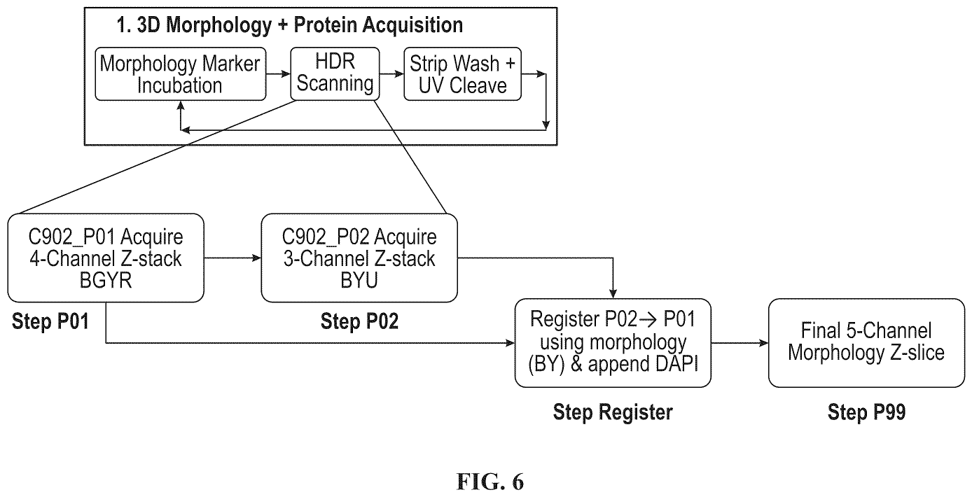

In some embodiments, the systems, media, and methods disclosed herein further comprise a multi-step image acquisition process. In some cases, the multi-step image acquisition process can be performed to avoid signal loss. In some embodiments, the systems, media, and methods disclosed herein can comprise a software module configured to reduce the 3D scan image to a 2D image. In some cases, reducing the 3D scan image to a 2D image can obtain the optimal focus region in a z-slice. In some embodiments, the systems, media, and methods disclosed herein can comprise a software module configured to deconvolve the 2D images. In some cases, the deconvolution of the 2D images can provide the optimal focus area for the images while retaining the z position of the images in their 3D scan image stack In some embodiments, the deconvolution of the 2D images can provide the optimal clarity for the images while retaining the z position of the images in their 3D scan image stack. As illustrated in the non-limiting example of FIG. 6, two step image acquisition P01 and P02 were performed to avoid signal loss from DAPI acquisition using UV light. In Step P01, the system acquired the four channels of B, G, Y, R of z-stacks of 3D High Dynamic Range (HDR) across the tissue for FOVs of choice. In Step P02 of FIG. 6, for example, the system separated DAPI from the other two morphology channels, for registration with the previous step P01 to a 3D coordinate space. Once the P01 step was complete, for example, the FOV locations were revisited to acquire a new Z-stack with DAPI and two additional morphology channels, for example B and Y. The B and Y morphology channels, in one non-limiting example, were required to register the P01 and P02 z-stacks to the same 3D coordinate space, as shown in Step Register of FIG. 6. In the non-limiting example, once the stacks were aligned, they were combined to generate a complete 5-channel Z-stack as shown in Step P99 of FIG. 6. In some embodiments, once the morphology scan is performed, protein images can be acquired for all FOVs followed by the RNA acquisition. In some embodiments, in each cycle, images can be registered to the same 3D coordinate space using fiducial markers that can be placed on the tissue. In some embodiments, the systems, media and methods disclosed herein can comprise acquisition of timelapse images for the sample of interest using the multi-step image acquisition process.

### Image Enhancement and Bleaching Correction

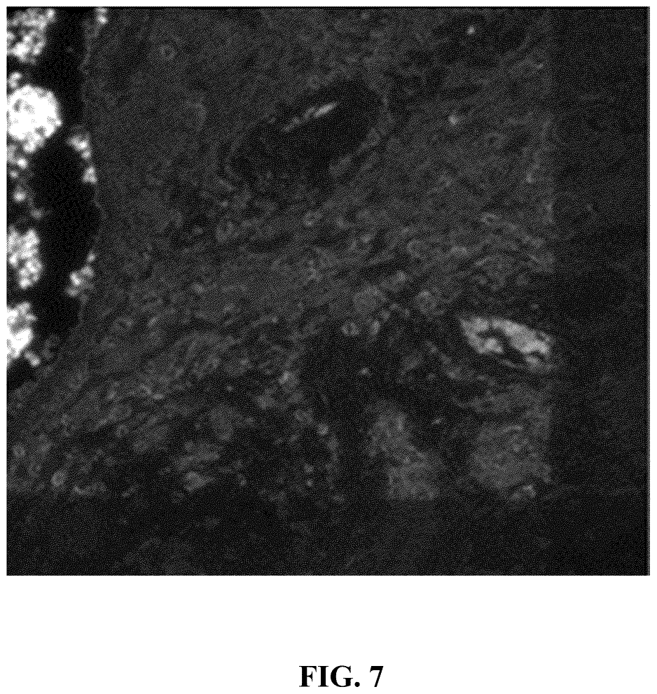

In some embodiments, photo-cleavable markers are used to obtain an unlimited set of source input channels by the process of repeat re-staining and clearing of markers using UV illumination and chemical wash in the tissue. In some cases, repeat scanning of tissue areas an introduce some photo bleaching and/or dimming at the edge of the FOV. In one non, limiting example, FIG. 7 shows darkening bands on the right and bottom sides of the image that degrade the signal and detectability of cells in those regions. In some cases, the closer the distances between FOVs and the larger number of repeated acquisitions cycles, the larger the effect of the photo-bleaching as displayed in the non-limiting example of FIG. 7.

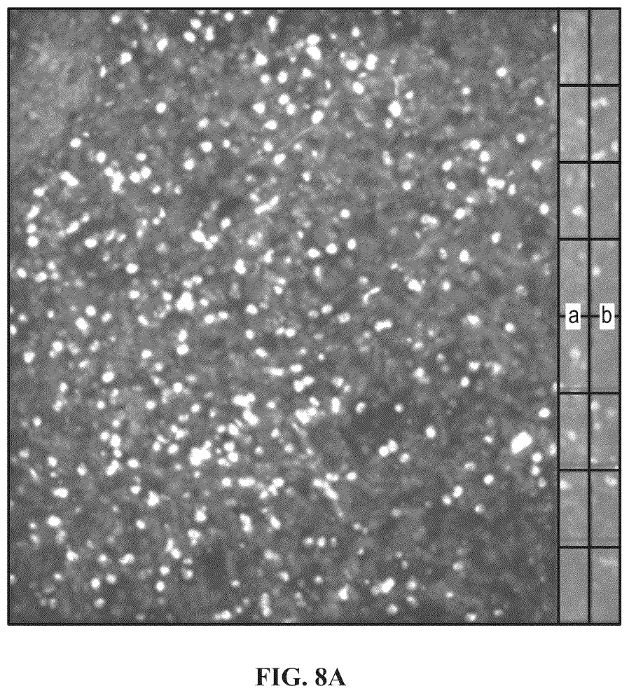

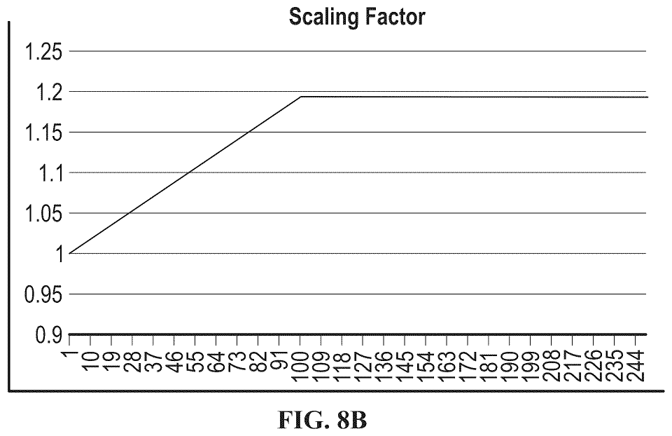

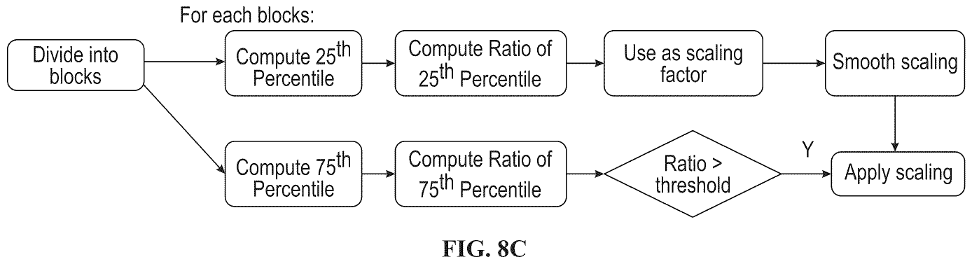

In some embodiments, the systems, media, and methods disclosed herein can further comprise normalizing the intensity of dimming regions using the nearest un-bleached portion of the image to correct edge artifacts. In some embodiments, measurements can be taken where the bleaching occurred by down sampling the image and dividing it into smaller blocks, as shown in, for example, FIG. 8A. In some embodiments, a transition smoothing function can be applied during the scaling calculation as shown in, for example, FIG. 8B. In one non-limiting example, the scheme of Normalization and scaling factor computation is illustrated in FIG. 8C. In some embodiments, at each block, the lower 25th percentile and top 75th percentile values can be computed, as shown in FIG. 8C. In some embodiments, the ratios of the lower 25th percentile and top 75th percentile can be computed, as shown in FIG. 8C. In some embodiments, the lower intensity quantile ratio can be used to normalize the intensity drop at that location by applying the scaling factor shown in, for example FIG. 8B. In some embodiments, the upper quantile can be used to determine the high intensity value of each block. In some embodiments, the sudden drop of this high intensity quantile between neighboring blocks can be used to determine the position where the bleaching starts. In some embodiments, the systems, media, and methods disclosed herein can further comprise applying bleaching correction to morphology and protein image acquisition.

In some embodiments, images can be corrected by conventional image enhancement techniques including denoising, deblurring, or image sharpening, or any combination thereof. In some embodiments, one or more denoising algorithms can be applied to the one or more images. The one or more denoising algorithms include filter based (Wiener, Gaussian, Median), using wavelet transforms, total variation (TV) denoising and deep learning-based approaches. Deblurring algorithms include the nearest-neighbor deconvolution, Richardson-Lucy deconvolution and total variation (TV) regularization, or any combination thereof. In some embodiments, image enhancement algorithms can include histogram equalization, contrast limited adaptive histogram equalization (CLAHE), contrast stretching, gamma correction, unsharp masking, or any combination thereof. In some embodiments, Nearest Neighbor Deconvolution can be used. In some cases, Nearest Neighbor Deconvolution can comprise eliminating blur signals by utilizing adjacent z-planes. In some embodiments, Nearest Neighbor Deconvolution does not require point spread function (PSF) data. In some embodiments, Nearest Neighbor Deconvolution can be performed for the correction on an image at location z, I, using images from the previous and next z locations I_{zprev} and I_{znext} as described below.

I_{decon}=1−A*(G(I_{zprev},0)+G(I_{znext}>σ))

Wherein A is the multiplier factor 0-1. G=Gaussian kernel, and σ is determined from optical numerical aperture (NA) and sampling rate in z (δ_{z}).

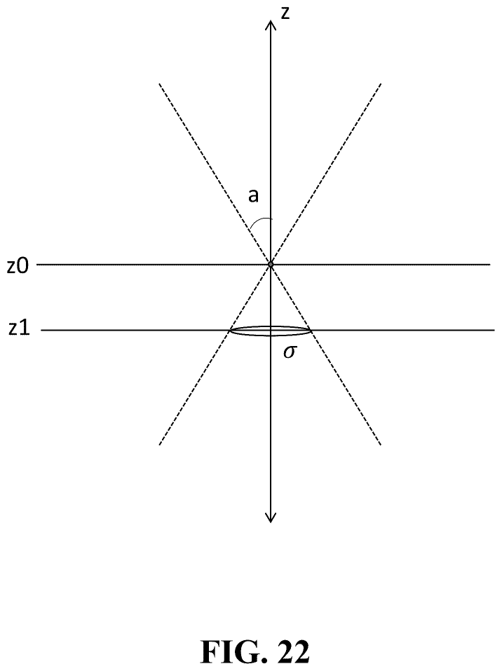

In some embodiments, the relationship between NA and the width of the blurring function can be described using the objective lens cone angle as NA=n sin(a) as illustrated in FIG. 22, where n is the refractive index. Therefore, in some embodiments, a=arcsin(NA/n). In some non-limiting examples, using a water-based objective, n=1.33333 and NA=1.10, it can be derived that a=55.59 deg. Using, for example, FIG. 22, gaussian kernel width can be derived as σ=δ_{z} tan(a).

### RNA Spot Detection and Density Map Generation

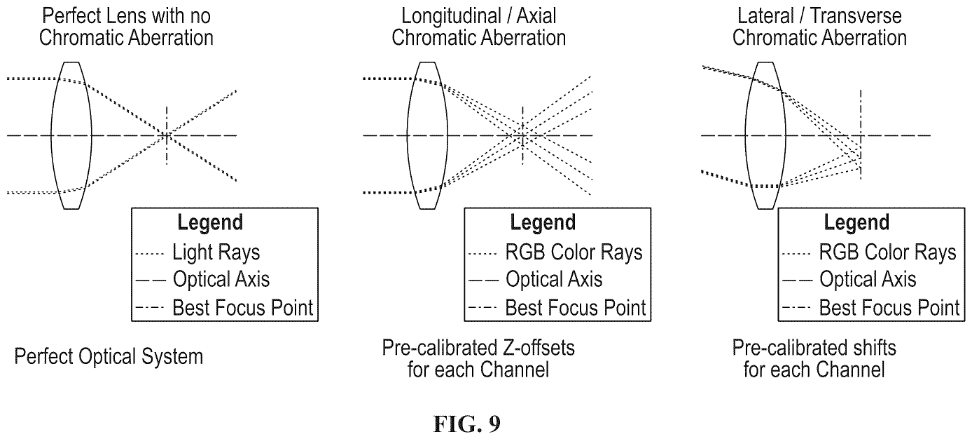

In some embodiments, RNA molecules can be detected using single molecule fluorescence barcodes, which appear in the image as visible spots. In some embodiments, the combination of spots can correspond to certain RNA transcripts as encoded in the chemical reagents used. In some embodiments, the systems, media, and methods disclosed herein can further comprise applying the location and density of raw spots to infer additional cell morphology. In some embodiments, the systems, media, and methods disclosed herein can further comprise applying the location and density of raw spots to augment additional cell morphology. In some embodiments, each RNA spot can be detected from image slices acquired across the tissue section. In some embodiments, the detection can use the Laplacian of Gaussian (LoG) bandpass filter. In some embodiments, the LoG bandpass filter can be designed to match the morphology of reporter signal in each channel. In some embodiments, the precise location of the spots can be estimated using a 2D paraboloid surface fit. In some embodiments, chromatic aberration of the optics can be accounted for ensuring spatial alignment between each channel. In some embodiments, the lens does not focus all wavelengths (channels) to the same point. In one non-limiting example, FIG. 9 illustrates the aberration generated in the axial direction (middle graphic) and the lateral direction (right graphic), comparing to the perfect lens on the left. In some embodiments, the systems, media, and methods disclosed herein can further comprise applying unique calibrations that determine the z-offsets for each wavelength during acquisition to correct for axial chromatic aberrations.

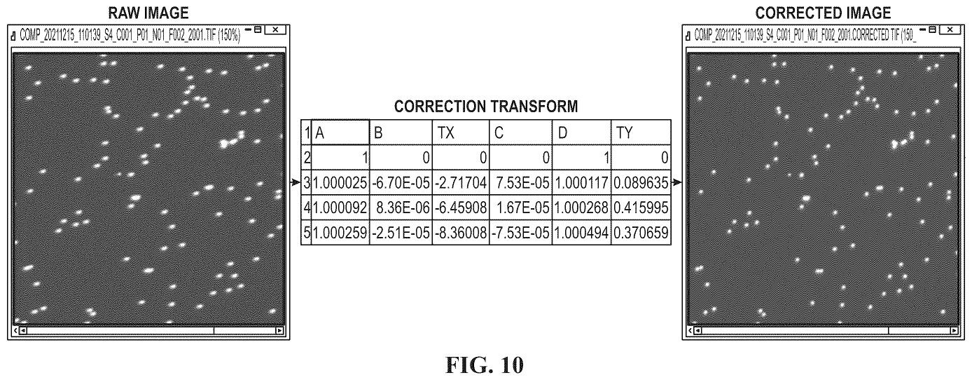

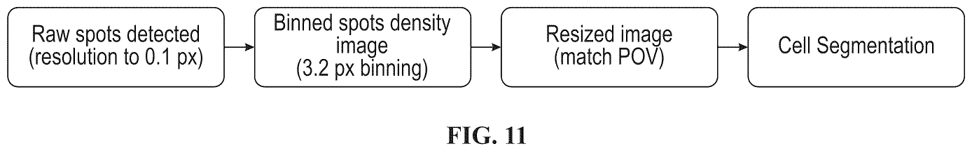

In some embodiments, the systems, media, and methods disclosed herein can further comprise performing the lateral chromatic aberration correction on each multi-channel image. In some embodiments, performing the lateral chromatic aberration correction can be performed using a precalibrated transform. In some non-limiting examples, as shown in, for example, FIG. 10, there are over 10M high-confidence (high signal) spot locations detected for each FOV that are resolved to a tenth of a pixel (−12 nm). As shown in, for example, FIG. 10, when the high-density spots were plotted in image space, they coincided with cell structures and can be used to infer and augment the cell morphology. In some embodiments, the spot locations can be binned into a lower-resolution pixel space. As shown in, for example, FIG. 10, this binning creates a detailed morphology image that can be successfully segmented using the cell segmentation pipeline. In some embodiments, the systems, media, and methods disclosed herein can further comprise applying binned 2D histograms of spot locations as an additional input segmentation channel. As shown in FIG. 11, as a non-limiting example, raw spots were detected with a resolution of 0.1 of a pixel, to form a detailed morphology density image. In some non-limiting examples, spots for each FOV were resolved to a tenth of a pixel, as illustrated in Table 1. As a non-limiting example, X and Y were in decipixels with a range [0, 42560].

TABLE 1
Example of spatial coordinates for spots detected.

| | X | Y |
|---|---|---|
| Spot 1 | 37322 | 916 |
| Spot2 | 37240 | 1020 |
| Spot 3 | 35484 | 1768 |
| Spot4 | 17510 | 5530 |
| Spot 5 | 17463 | 5598 |

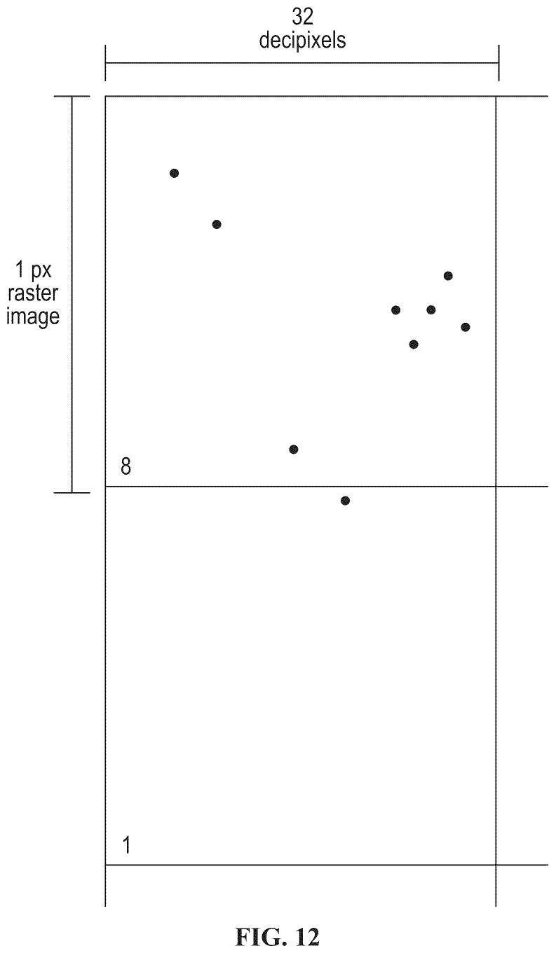

As shown in, for example, FIG. 12, a grid 1330×1330 was overlaid on the range [0, 42560]. For example, the total number of spots falling into each box of the grid were tallied to determine the grayscale value in the resulting spots density map image. For example, the density map image was resized to the FOV dimensions of 4256×4256, reducing noise in the density map image. For example, the image was then used in the cell segmentation workflow as a standard morphology channel obtained with fluorescence staining.

### Multi-Modal Cell Segmentation

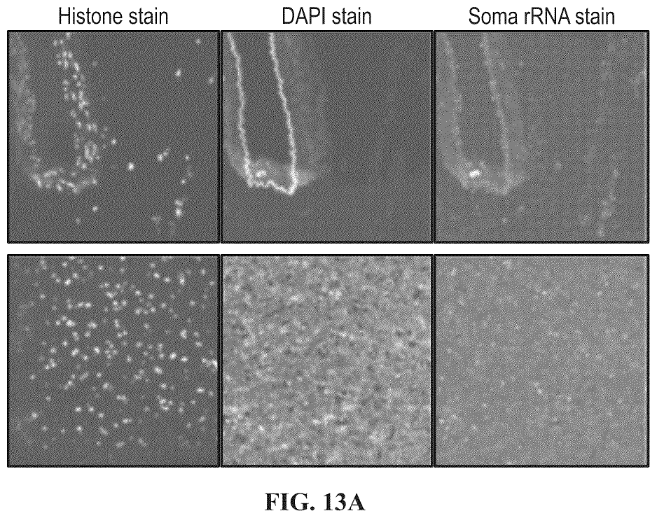

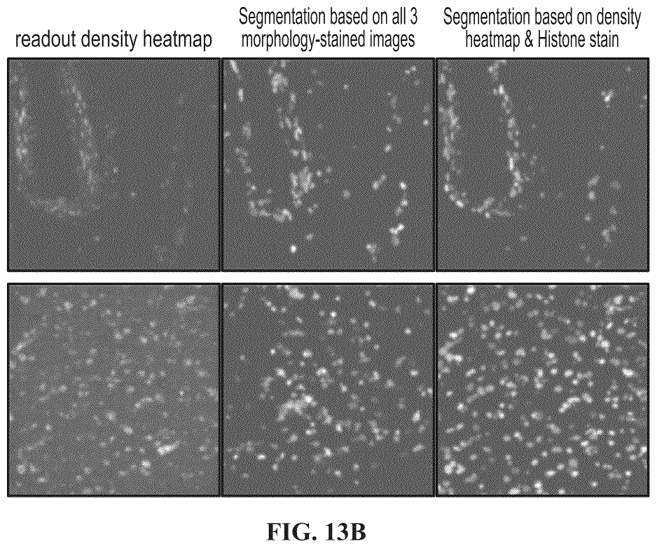

In some embodiments, the systems, media, and methods disclosed herein can comprise retrieving five morphological channels. In some embodiments, retrieving five morphological channels can express more descriptions of cell boundaries than using the nuclear channel alone. In some embodiments, the systems, media, and methods disclosed herein can comprise retrieving 64 or more different protein channels. In some embodiments, about 1, 2, 3, 4, 5, 10, 20, 30, 40, 50, 60, 70, 80, 90, 100, or more than 100 different protein channels can be retrieved. FIG. 13A, for example, shows several protein channels including a channel for Histone stain, DAPI stain, and Soma rRNA stain. FIG. 13B, for example, shows several segmentation processed channels, including a readout density heatmap, segmentation based on all 3 morphology-stained images, and segmentation based on density heatmap and histone stain.

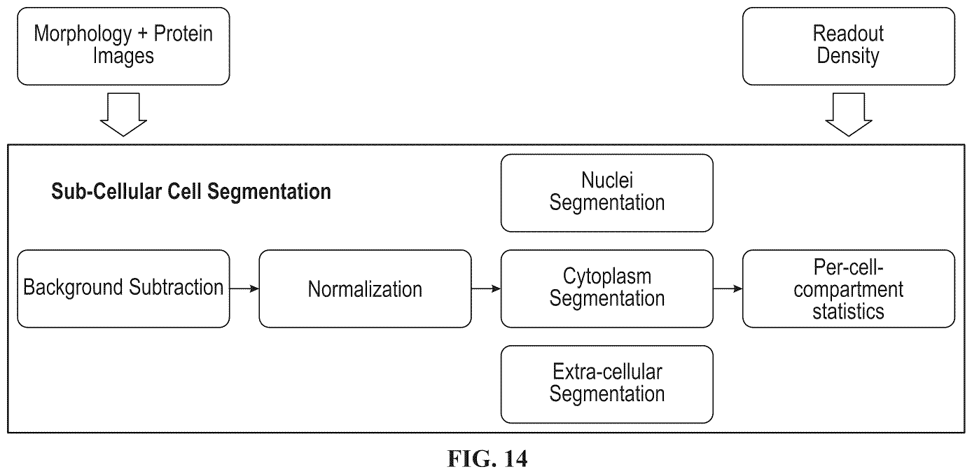

In some embodiments, the number of different protein channels retrieved can be dictated by the number of protein markers, scanning cycling parameters, additional transcript-based information using a spot density map, or any combination thereof, as illustrated in FIG. 14. In some embodiments, the systems, media, and methods disclosed herein can comprise combining the morphology and protein images with the same morphology type into smaller sets of input channels. In some embodiments, each protein can be annotated to describe its morphology. In some embodiments, the annotations can include cytoplasm or nuclear expression, or both. In some embodiments, the annotation can describe different cell type expressions. In some cases, the cell type expression can comprise Glia, Astrocytes and other neuronal cell types, or any combination thereof. In some embodiments, these annotations can be used to combine each protein image together based on cells having the same morphology or same cell type, or both. In some embodiments, a projection method can be used to collapse multiple protein images with the same morphology into the 5-channel images. In some embodiments, the projection method can be one or more of maximum intensity projection, principal component analysis, singular value decomposition, multi-reference alignment, density map averaging, or any combination thereof. In some embodiments, the readout or spot density image, or both can be added as an additional channel. In some embodiments, all input channels can be background subtracted and normalized prior to a segmentation step. In some embodiments, cell segmentation can be one or more of nuclei segmentation, cytoplasm segmentation or extra-cellular segmentation, or any combination thereof. In some embodiments, outputs from each cell segmentation can be combined as the final segmentation outputs. In some embodiments, per-cell and per-compartment statistics can be performed.

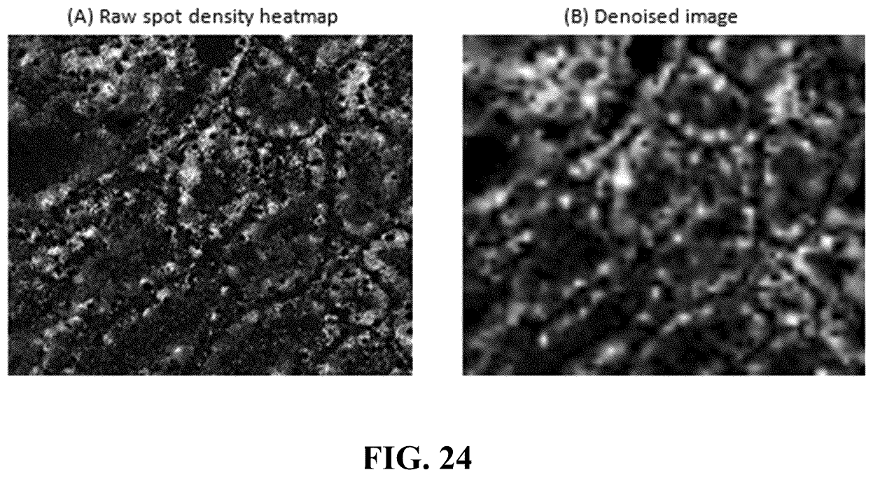

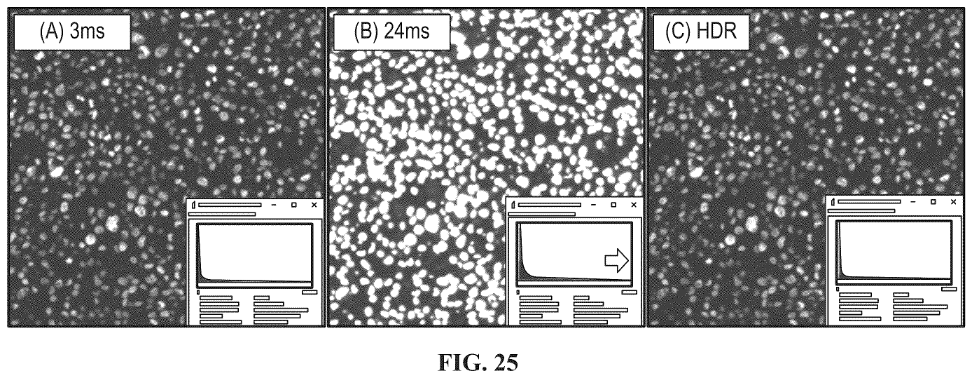

In some embodiments, image preprocessing to minimize imaging artifacts and improve the quality of images can be applied to raw images before processing by cell segmentation modules. In some embodiments, transcriptional spot density heatmaps can be treated processed using image preprocessing techniques. In some embodiments, the preprocessing can be used to achieve better signal to noise ratio between intracellular and extracellular regions. One non-limiting example of denoising preprocessing used on spot density heatmaps is shown in FIG. 24. In some embodiments, spot density heatmaps can be used without further preprocessing. In some embodiments, the image preprocessing step can employ a combination of conventional image processing techniques, such as one or more of deconvolution, gaussian blur, normalization, feature extraction, Fourier transforming, linear filtering, contrast enhancement, binarization, or any combination thereof. In some embodiments, the image preprocessing step can utilize machine learning methods, for example Noise2Self and cellpose 3.0 image restoration models.

In some embodiments, to provide sub-cellular segmentation, a nuclear channel can be segmented. In some embodiments, the nuclear channel can be segmented in addition to the other channels containing membrane and cytoplasmic content. In some embodiments, multi-step segmentation produces sub-cellular results defining the separation of nuclei and cytoplasmic regions. In some embodiments, overlap between cells can be measured using the Intersection over Union (IoU) score. For example, in some embodiments, when the IoU score is low, the overlapping region can be assigned to the segment with a higher confidence signal. In some embodiments, nuclei channels may have a stronger signal. In some cases, low intersection pixels can be assigned as nuclear segments. In some embodiments, the nuclear segments are not cytoplasm. In some embodiments, when the intersection is high, the two segmentation results can be merged to combine both nuclei and cytoplasm. In some embodiments, the segmentation system produces masks that define compartments. In some cases, the compartments can be defined as nuclei or cytoplasm. In some embodiments, the compartments can be defined as areas of each cell detected in an image. In some embodiments, measurements and statistics can be performed for each cell by using these masks. In some embodiments, specific membrane and extra-cellular regions can provide biological information.

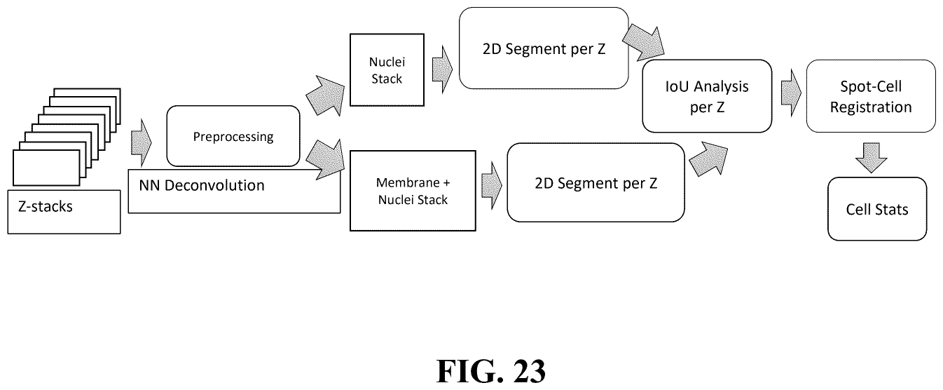

In some embodiments, the Intersection of Union (IoU)-based combination approach, as illustrated in FIG. 23, for example, can be employed to combine multiple segmentation outcomes. In some embodiments, the IoU-based combination approach can be performed in a step-wise manner. In some embodiments, performing the IoU-based combination in a step-wise manner can combine multiple modalities of morphology in achieving the final outcome. In some embodiments, the IoU-based combination approach can combine 2D cell segmentation outcomes of individual 2D images into the 3D cell segmentation volumetric outcomes of the corresponding 3D scan image stack. In some embodiments, the correlation can comprise labelling each cell in all z-locations using maximum IoU scores for mapping consistently. In some embodiments, other measures such as cell focus profile across z-slices can be used to determine cell label continuity through the z-slices. In some embodiments, analyzing centroid and shape changes of cells across the z slices can be used to label cells in 3D stack. In some embodiments, 3D stack can be directly segmented using a machine learning model that takes a 3D stack input. In some embodiments, as shown in FIG. 23, extending 2D to 3D in this arrangement can comprise performing multi-modal segmentation in 2D for each z-slice followed by 3D cell label stitching to correlate cellids in each z slice, forming 3D cell labels. FIG. 23 illustrates a non-limiting example of a 3D segmentation pipeline.

In some embodiments, localization of objects inside cells, like nucleus, can be mapped to the cell body contour using maximum IoU scores. In some embodiments, localization of cells in different source images, like the images taken at different z coordinates or time points, can be mapped to each other using maximum IoU scores. In some embodiments, the localization points of each of the different source images taken at different z coordinates or time points that, when matched, obtain the maximum IoU scores, can be mapped together to form a stacked image. In some embodiment, the spot images can be registered to the same reference as the morphology stained images. In some embodiments, timelapse images can be taken for the sample of interest. In some embodiments, the IoU-based combination approach can be applied to combine cell segmentation outcomes of same sample at different time points to track the behavior of same cells over time.

In some embodiments, extra-cellular regions can be detected. In some embodiments, extra-cellular regions can be included as part of segmentation results. In some cases, extra-cellular regions can comprise one or more structures of the extracellular space of, for example, the brain. In some cases, extracellular structures can comprise one or more vesicles. In some embodiments, each connected component can be detected and masked. In some embodiments, masking can be performed using auto-thresholding techniques. In some embodiments, auto-thresholding techniques can comprise finding high intensity regions in the input morphology channel or using neural network models that have been trained to detect cell membrane or specific extra-cellular regions.

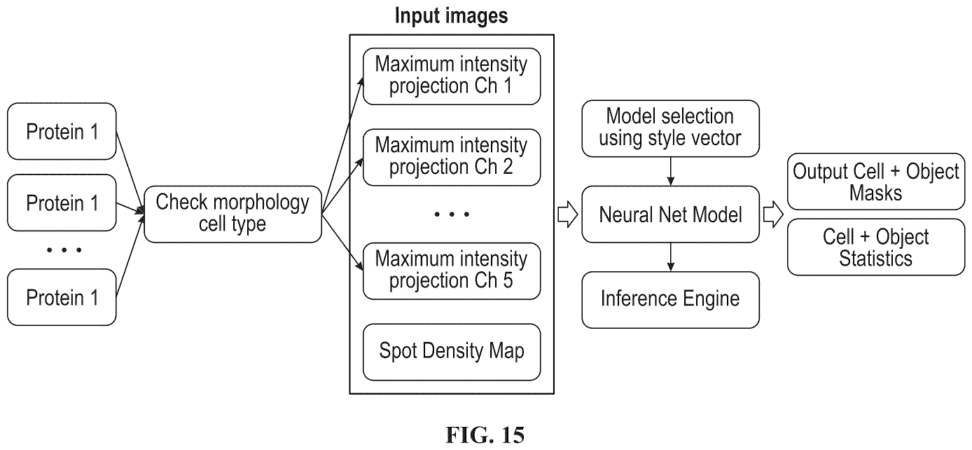

In one non-limiting example, as illustrated in FIG. 15, all protein expressing microglia cell type were combined with the microglia morphology channel using an Ibal marker. As illustrated in FIG. 15, the input image channels included morphology channels and a channel of spot density map for the same biological sample. In some embodiments, the maximum intensity pixel values can be projected for all input morphology channels. In some embodiments, the readout or spot density image, or both, can be added as a separate channel. In some embodiments, the nuclei, cytoplasm, or extra-cellular segmentation can be performed for each feeding channel. In some embodiments, a cell segmentation algorithm can be selected based on one or more feeding channels. In some embodiments, cell segmentation can be based on one or more algorithms. In some cases, the one or more algorithms can comprise Cellpose Deep Learning algorithm, adaptive thresholding, Watershed algorithm, thresholding, region-based segmentation, active contours, Convolutional Neural Networks (CNN), Level Set Methods, Graph-based algorithms, fuzzy c-means clustering, Deep watershed, image morphology, or Markov Random Fields, or any combination thereof. In some embodiments, nuclei segmentation, cytoplasm segmentation and extra-cellular segmentation can be based on the same algorithm. In some embodiments, nuclei segmentation, cytoplasm segmentation, and extra-cellular segmentation can be based on different algorithms. As shown in, for example, FIG. 15, selected algorithms were used for neural network models, and models were generated using ground-truth datasets. In some embodiments, a model can be selected to fit a specific input morphology by comparing the image and model style vectors. In some embodiments, after neural network inference, outputs can be generated by measuring image property within each cell or cell compartment mask area. In some embodiments, outputs from all models can be combined as one final output. In some embodiments, per-cell or per-compartment statistics, or both, is performed.

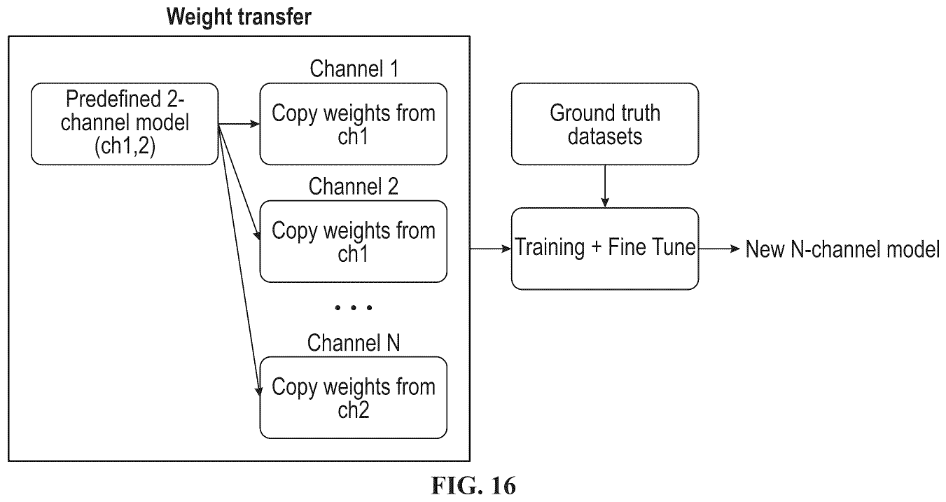

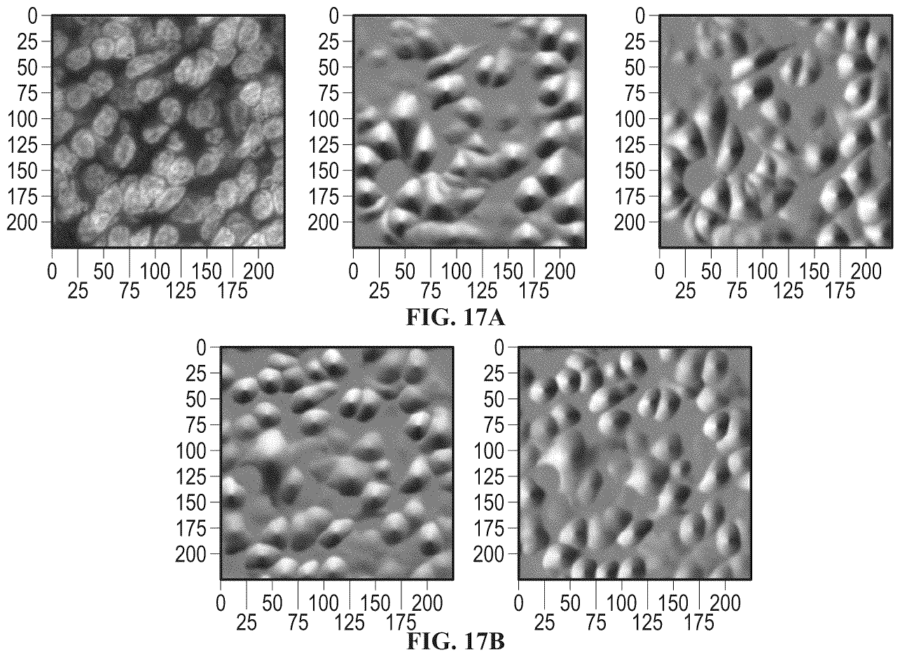

In some embodiments, the systems, media, and methods disclosed herein can comprise multiple input channels in the model. FIG. 16 illustrates a non-limiting example for generating a new N-channel model to adapt to the number of new morphology channels. In some embodiments, the systems, media, and methods disclosed herein can comprise applying the transfer learning technique. In some embodiments, the transfer learning technique can be applied to reuse pre-existing trained deep learning model weights in higher dimensions. In some embodiments, as shown in the weight transfer block in FIG. 16, where a predefined 2-channel model was used as a starting model, new channels can be added by copying the weights from the original 2 channels. In some embodiments, the new model can be trained based on the new defined weights and ground truth datasets. In some embodiments, the new N-channel model can then be generated. Various new 2-Channel and 5-Channel outputs are shown by FIGS. 17A-17B, with FIG. 17A showing non-limiting examples of new 2-Channel models, and FIG. 17B showing non-limiting examples of new 5-Channel models.

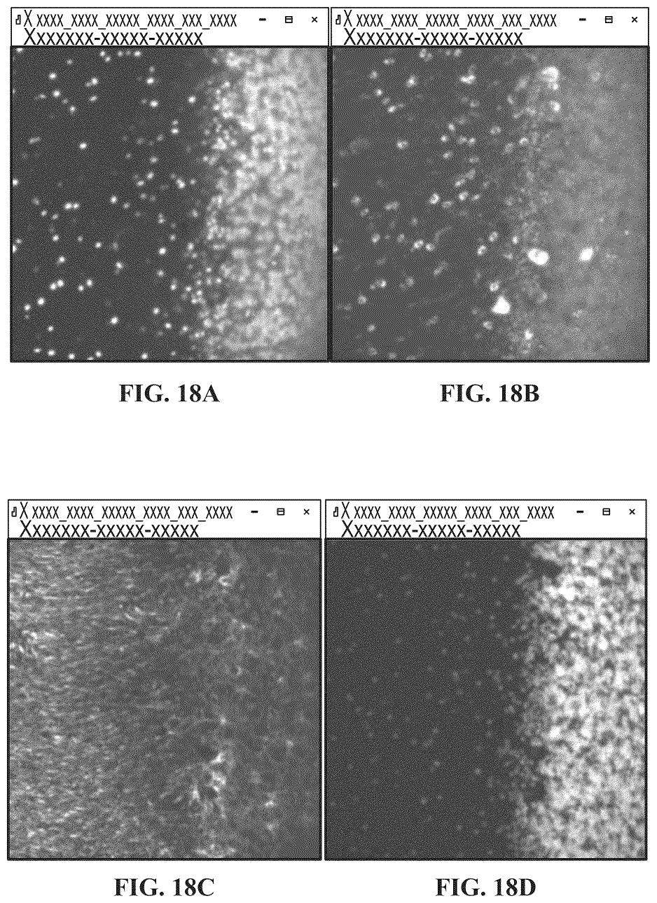

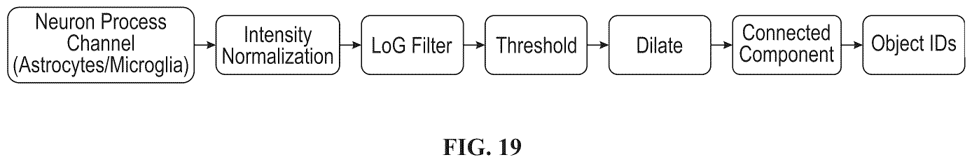

In some embodiments, separate nuclei only segmentation and cytoplasm segmentation can be combined. In some embodiments, the combination of nuclei segmentation and cytoplasm segmentation can ensure segmentation output for tissue with complete sets of nuclei and membrane signals. In some embodiments, the combination of nuclei segmentation and cytoplasm segmentation can ensure segmentation output for areas where the membrane signals are weak. In some embodiments, segmenting both nuclei and cytoplasm can provide subcellular resolution and precision. As illustrated in non-limiting examples FIGS. 18A-18D, nuclei expressed images were selected as input for nuclei segmentation, and all morphology input channels including nuclei were fed for cytoplasm segmentation. As shown in FIGS. 18A-18D, results from nuclei and cytoplasm segmentations were combined by analyzing overlaps and intersections (IoU) between segmentation results. Various markers were applied to the cell segmentation results, for example, FIG. 18A shows neuron cells with nuclei marker HistoneH3 applied, FIG. 18B shows neuron cells with rRNA membrane markers applied, FIG. 18C shows astrocytes with GFAP markers applied, and FIG. 18D shows nuclei with DAPI markers applied.

In some embodiments, the systems, media, and methods disclosed herein can further comprise segmentation of extra-cellular processes as a third segmentation step. In some embodiments, extra-cellular regions can be structures that exist in the image but do not contain nucleic information. In some embodiments, the systems, media, and methods disclosed herein can further comprise tracing each of one or more neuronal processes using a filter. In some embodiments, the filter can comprise Laplacian of Gaussian (LoG) filter. In some embodiments, the filter can be followed by a connected component algorithm to separate detected processes into individual components, as illustrated in FIG. 19. In some embodiments, the LoG filter can extract thin neuron processes from normalized brain-specific markers. In some cases, brain-specific markers can comprise an astrocytes or microglia, or both, input channel. In some embodiments, a threshold value can be applied to the LoG output to create a mask. In some embodiments, the mask is then dilated to match the size of the neuron. In some embodiments, each protrusion can be detected using standard thresholding techniques. In some cases, the standard thresholding techniques can include Otsu, Moments, Li, Huang and Bernsen local thresholding methods, or any combination thereof. In some embodiments, each connected neuron can be numbered using a connected component algorithm. In some embodiments, the connected component algorithm can be used to number cells segmented from the image. In some embodiments, each protrusion can be detected using machine learning models trained to detect cellular structures.

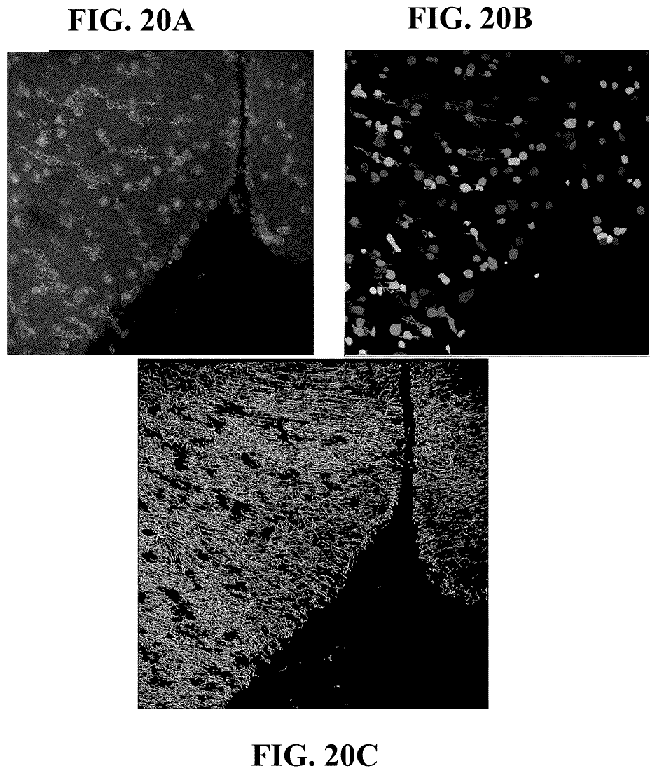

In some embodiments, processes that fully encapsulate cell soma can be merged with the nuclei segments and marked as cells. In some embodiments, processes that have no connections to the cell body can be marked as objects, as shown in FIGS. 20A-20C. For example, FIG. 20A shows an overlay of cells and extracellular processes that were masked as shown in FIG. 20B to isolate the extra-cellular segments as shown in, for example, FIG. 20C. In some embodiments, the merging between cell protrusions and nuclei can be performed using a machine learning algorithm trained to perform merging operations.

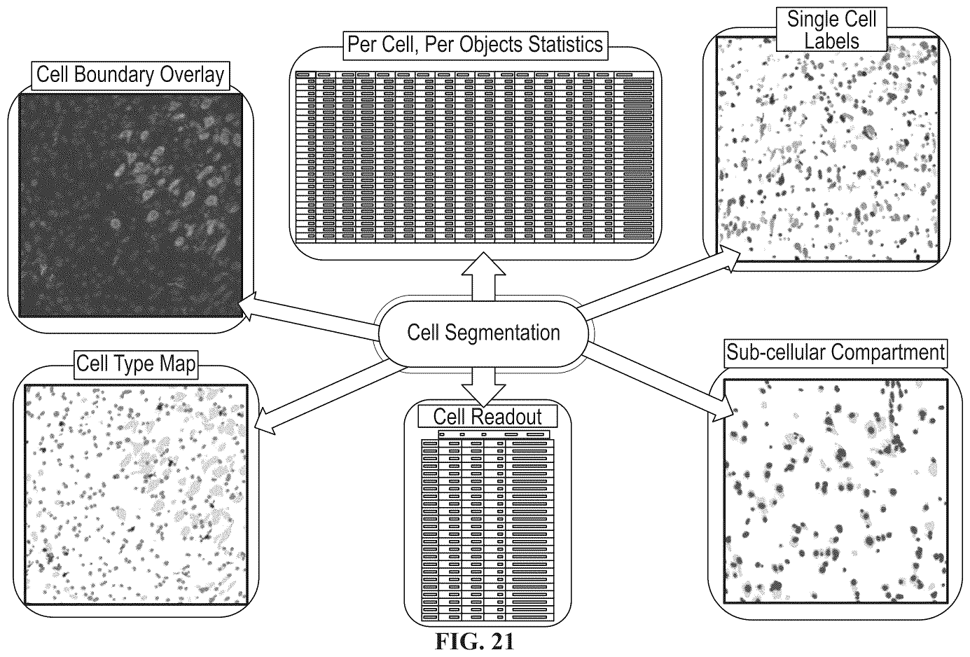

In some embodiments, because each neuron process can be individually identifiable as one or more objects, transcriptomic, cell typing, and other spatial data analysis can be performed in the same manner as single cell analysis. In some embodiments, all three segmentation steps can produce single cell labels using cell measurements and statistics, as well as per-object measurements. In some embodiments, cell labels can be further split into nuclei and cytoplasm compartments. In one non-limiting example for brain tissue, each individual cell type was marked in different channels and were identified as shown in FIG. 21.

### Per-Cell Statistics

In some embodiments, the segmentation output can provide statistics for each detected cell. The statistics can include statistics describing fluorescence properties, spatial locations, or features, or any combination thereof. In some embodiments, these properties can comprise average, median, and maximum fluorescent intensities. In some embodiments, these properties can comprise various shape descriptors. In some cases, shape descriptors can comprise circularity, compactness, aspect ratio, convexity, solidity, eccentricity and perimeter. These shape descriptors are described below:

$$Aspect Ratio = \frac{{W}_{bb}}{{H}_{bb}} .$$

Wherein W_{bb}=width of the bounding box and H_{bb}=height of the bounding box.

In some embodiments, to restrict the value of aspect ratio to 0-1, width can be assigned to be the shorter value of the bounding box size. In some embodiments, to restrict the value of aspect ratio to 0-1, height can be assigned to be the longer value of the bounding box size.

$$Circularity = \frac{4 * π * A}{{P}_{conv} * {P}_{conv}}$$

Wherein A is the area of the cell and P_{conv} is the convex perimeter to avoid concave irregularities.

$$Compactness = \frac{4 * π * A}{P * P} .$$

Wherein A is the area of the cell and P is regular cell perimeter.

$$Convexity = \frac{{P}_{conv}}{P} \\ Eccentricity = \frac{{L}_{minor}}{{L}_{major}} .$$

Wherein Lₘᵢₙₒᵣ is the length of the minor axis and Lₘₐⱼₒᵣ is the length of the major axis.

Solidity=\frac{A}A_{conv}}.

Wherein A is the area of the cell and A_{conv} is the convex area.

In some embodiments, metrics can be computed for every compartment, including nuclei and cytoplasm compartments. In some embodiments, these properties can be utilized to analyze cell sub-populations. In some embodiments, other properties such as texture can also be incorporated.

In some embodiments, when a small section of a tissue sample is imaged, some cells may be split across different field of views (FOV)s. In some embodiments, when used in the context of single cell transcriptomic analysis, these partial cell segments at the FOV intersections can create incomplete masks and produce incorrect analysis. In some embodiments, segmentation output measurements can identify cells that are split at the image boundary so that they can be filtered out downstream.

### Machine Learning

In some embodiments, the systems, media, and methods disclosed herein can comprise subcellular segmentation further comprising one or more deep learning models. In some embodiments, one or more deep learning models can be trained using images of cells or tissues. In some embodiments, images of cells or tissues can comprise microscope-derived images, morphology images, or fluorescence images, or any combination thereof. In some embodiments, a deep learning model can be trained to segment a particular tissue type. In some embodiments, a deep learning model can be trained to restore image quality for better cell segmentation outcomes.

In some embodiments, the systems, media, and methods disclosed herein can comprise training a machine learning model or applying a machine learning model, or both. In some embodiments, the machine learning model may perform dimension reduction. In some embodiments, the dimension reduction can be performed through a nonlinear dimensionality reduction algorithm. In some embodiments, the nonlinear dimensionality reduction algorithm may comprise Sammon's mapping, Principal curves and manifolds, Laplacian eigenmaps, Isomap, Locally-linear embedding, Local tangent space alignment, Maximum variance unfolding, Gaussian process latent variable models, t-distributed stochastic neighbor embedding, Relation perspective map, Contagion maps, Curvilinear component analysis, Curvilinear distance analysis, Diffeomorphic dimensionality reduction, Manifold alignment, Diffusion maps, Local multidimensional scaling, Nonlinear PCA, Data-driven high-dimensional scaling, Manifold sculpting, RankVisu, Topologically constrained isometric embedding, Uniform manifold approximation or projection (UMAP), or any combination thereof. In some embodiments, the UMAP algorithm may apply a feed-forward neural network on a subset of the data. In some embodiments, the UMAP algorithm may project the manifold clustering onto the entire dataset. In some embodiments, the UMAP algorithm may be a feed-forward algorithm. In some embodiments, the feed-forward neural network may be trained to approximate the identity function. In some embodiments, approximating the identity function can comprise mapping from a vector of values to the same vector. In some embodiments, the feed-forward neural network may be used for dimensionality reduction. In some embodiments, one of the hidden layers in the network may be limited to contain only a small number of network units.

In some embodiments, the systems, media, and methods disclosed herein can comprise training a machine learning model or applying a machine learning model, or both. In some embodiments, the machine learning model may comprise an unsupervised machine learning model, a supervised machine learning model, semi-supervised machine learning model, or a self-supervised machine learning, or any combination thereof. In some embodiments, the supervised machine learning model may comprise, for example, a Random Forest, a support vector machine (SVM), a neural network, or a deep learning algorithm, or any combination thereof.

In some embodiments, the platforms, systems, media, and methods disclosed herein can comprise a machine learning model utilizing one or more neural networks. In some embodiments, a neural network can learn the relationships between an input dataset and a target dataset. In some embodiments, a neural network may be a software representation of a human neural system. In some embodiments, the human neural system may be the cognitive system. In some embodiments, the neural network capture “learning” and “generalization” abilities as used by a human. In some embodiments, the machine learning algorithm can comprise a neural network comprising a CNN. In some embodiments, machine learning algorithm structural components can comprise one or more of: CNNs, recurrent neural networks, dilated CNNs, fully-connected neural networks, deep generative models, transformers, or Boltzmann machines, or any combination thereof.

In some embodiments, a neural network can comprise a series of layers termed “neurons.” In some embodiments, a neural network can comprise an input layer, to which data is presented; one or more internal, and/or “hidden,” layers; and an output layer. In some embodiments, a neuron may be connected to neurons in other layers via connections that have weights, which are parameters that control the strength of the connection. In some embodiments, the number of neurons in each layer may be related to the complexity of the problem to be solved. In some embodiments, the minimum number of neurons required in a layer may be determined by the problem complexity, and the maximum number may be limited by the ability of the neural network to generalize. In some embodiments, the input neurons may receive data being presented and then transmit that data to the first hidden layer through connections' weights, which are modified during training. The first hidden layer may process the data and transmit its result to the next layer through a second set of weighted connections. In some embodiments, each subsequent layer may “pool” the results from the previous layers into more complex relationships. In some embodiments, whereas conventional software programs require writing specific instructions to perform a function, neural networks are programmed by training them with a known sample set and allowing them to modify themselves during (and after) training so as to provide a desired output such as an output value. In some embodiments, after training, when a neural network is presented with new input data, it is configured to generalize what was “learned” during training and apply what was learned from training to the new previously unseen input data in order to generate an output associated with that input.

In some embodiments, the neural network can comprise artificial neural networks (ANNs). In some embodiments, ANNs may be machine learning algorithms that may be trained to map an input dataset to an output dataset, where the ANN comprises an interconnected group of nodes organized into multiple layers of nodes. For example, the ANN architecture may comprise at least an input layer, one or more hidden layers, and an output layer. In some embodiments, the ANN may comprise any total number of layers, and any number of hidden layers, where the hidden layers function as trainable feature extractors that allow mapping of a set of input data to an output value or set of output values. As used herein, a deep learning algorithm (such as a deep neural network (DNN)) is an ANN comprising a plurality of hidden layers, e.g., two or more hidden layers. Each layer of the neural network may comprise a number of nodes (or “neurons”). In some embodiments, a node can receive input that comes either directly from the input data or the output of nodes in previous layers, and performs a specific operation, e.g., a summation operation. In some embodiments, a connection from an input to a node can be associated with a weight or weighting factor.

In some embodiments, the node may sum up the products of all pairs of inputs and their associated weights. In some embodiments, the weighted sum may be offset with a bias. In some embodiments, the output of a node or neuron may be gated using a threshold or activation function. In some embodiments, the activation function may be a linear or non-linear function. In some embodiments, the activation function may be, for example, a rectified linear unit (ReLU) activation function, a Leaky ReLU activation function, or other function such as a saturating hyperbolic tangent, identity, binary step, logistic, arctan, softsign, parametric rectified linear unit, exponential linear unit, softplus, bent identity, softexponential, sinusoid, sine, Gaussian, or sigmoid function, or any combination thereof.

In some embodiments, the weighting factors, bias values, and threshold values, or other computational parameters of the neural network, may be “taught” or “learned” in a training phase using one or more sets of training data. For example, the parameters may be trained using the input data from a training dataset and a gradient descent or backward propagation method so that the output value(s) that the ANN computes are consistent with the examples included in the training dataset.

The number of nodes used in the input layer of the ANN or DNN may be at least about 10, 50, 100, 200, 300, 400, 500, 600, 700, 800, 900, 1000, 2000, 3000, 4000, 5000, 6000, 7000, 8000, 9000, 10,000, 20,000, 30,000, 40,000, 50,000, 60,000, 70,000, 80,000, 90,000, 100,000, or greater than 100,000, including increments therein.

In other instances, the number of nodes used in the input layer may be at most about 100,000, 90,000, 80,000, 70,000, 60,000, 50,000, 40,000, 30,000, 20,000, 10,000, 9000, 8000, 7000, 6000, 5000, 4000, 3000, 2000, 1000, 900, 800, 700, 600, 500, 400, 300, 200, 100, 50, 10, or less than 10, including increments therein. In some instances, the total number of layers used in the ANN or DNN (including input and output layers) may be at least about 3, 4, 5, 10, 15, 20, or greater, including increments therein. In other instances, the total number of layers may be at most about 20, 15, 10, 5, 4, 3, or less, including increments therein.

In some instances, the total number of learnable or trainable parameters, e.g., weighting factors, biases, or threshold values, used in the ANN or DNN may be at least about 10, 50, 100, 200, 300, 400, 500, 600, 700, 800, 900, 1000, 2000, 3000, 4000, 5000, 6000, 7000, 8000, 9000, 10,000, 20,000, 30,000, 40,000, 50,000, 60,000, 70,000, 80,000, 90,000, 100,000, or greater than 100,000, including increments therein. In other instances, the number of learnable parameters may be at most about 100,000, 90,000, 80,000, 70,000, 60,000, 50,000, 40,000, 30,000, 20,000, 10,000, 9000, 8000, 7000, 6000, 5000, 4000, 3000, 2000, 1000, 900, 800, 700, 600, 500, 400, 300, 200, 100, 50, 10, or less than 10, including increments therein.

In some embodiments, the platforms, systems, media, and methods disclosed herein include a machine learning model can comprise a neural network such as a deep CNN. In some embodiments in which a CNN is used, the network can be constructed with any number of convolutional layers, dilated layers or fully-connected layers. In some embodiments, the number of convolutional layers can be between 1-10. In some embodiments, the number of dilated layers can be between 0-10. In some embodiments, the total number of convolutional layers (including input and output layers) may be at least about 1, 2, 3, 4, 5, 10, 15, 20, or greater, and the total number of dilated layers may be at least about 1, 2, 3, 4, 5, 10, 15, 20, or greater. In some embodiments, the total number of convolutional layers may be at most about 20, 15, 10, 5, 4, 3, or less, and the total number of dilated layers may be at most about 20, 15, 10, 5, 4, 3, or less. In some embodiments, the number of convolutional layers can be between 1-10 and the fully-connected layers between 0-10. In some embodiments, the total number of convolutional layers (including input and output layers) may be at least about 1, 2, 3, 4, 5, 10, 15, 20, or greater, and the total number of fully-connected layers may be at least about 1, 2, 3, 4, 5, 10, 15, 20, or greater. In some embodiments, the total number of convolutional layers may be at most about 20, 15, 10, 5, 4, 3, 2, 1, or less, and the total number of fully-connected layers may be at most about 20, 15, 10, 5, 4, 3, 2, 1, or less.

In some embodiments, a machine learning algorithm can comprise a neural network comprising a convolutional neural network (CNN), a recurrent neural network (RNN), dilated CNN, fully-connected neural networks, deep generative models or deep restricted Boltzmann machines, or any combination thereof.

In some embodiments, a machine learning model can comprise one or more CNNs. In some embodiments, the CNN may be deep and feedforward ANNs. In some embodiments, the CNN may be applicable to analyzing visual imagery. In some embodiments, the CNN may comprise an input, an output layer, and multiple hidden layers. In some embodiments, the hidden layers of a CNN may comprise convolutional layers, pooling layers, fully-connected layers and normalization layers. In some embodiments, the layers may be organized in 3 dimensions: width, height, and depth.

In some embodiments, the convolutional layers may apply a convolution operation to the input, and pass results of the convolution operation to the next layer. In some embodiments, for processing images, the convolution operation may reduce the number of free parameters, allowing the network to be deeper with fewer parameters. In some embodiments of neural networks, each neuron may receive input from some number of locations in the previous layer. In some embodiments in a convolutional layer, neurons may receive input from only a restricted subarea of the previous layer. In some embodiments, the convolutional layer's parameters may comprise a set of learnable filters (or kernels). In some cases, the learnable filters may have a small receptive field and extend through the full depth of the input volume. In some cases, during the forward pass, each filter may be convolved across the width and height of the input volume, compute the dot product between the entries of the filter and the input, and produce a two-dimensional activation map of that filter. In some embodiments, as a result, the network may learn filters that activate when it detects some specific type of feature at some spatial position in the input.

In some embodiments, a machine learning model can comprise an RNN. RNNs are neural networks with cyclical connections that can encode and process sequential data. In some embodiments, an RNN can include an input layer that is configured to receive a sequence of inputs. In some embodiments, an RNN may additionally include one or more hidden recurrent layers that maintain a state. In some embodiments, at each step, each hidden recurrent layer can compute an output and a next state for the layer. In some embodiments, the next state may depend on the previous state and the current input. In some embodiments, the state may be maintained across steps and may capture dependencies in the input sequence.

In some embodiments, an RNN can be a long short-term memory (LSTM) network. In some embodiments, an LSTM network may be made of LSTM units. In some embodiments, an LSTM unit may include of a cell, an input gate, an output gate, and a forget gate. In some embodiments, the cell may be responsible for keeping track of the dependencies between the elements in the input sequence. In some embodiments, the input gate can control the extent to which a new value flows into the cell, the forget gate can control the extent to which a value remains in the cell, and the output gate can control the extent to which the value in the cell is used to compute the output activation of the LSTM unit.

In some embodiments, the neural network can comprise an attention mechanism (e.g., a transformer). In some embodiments, attention mechanisms may focus on, or “attend to,” certain input regions while ignoring others. In some embodiments, this may increase model performance because certain input regions may be less relevant. In some embodiments, at each step, an attention unit can compute a dot product of a context vector and the input at the step, among other operations. In some embodiments, the output of the attention unit may define where the most relevant information in the input sequence is located. In some embodiments, the attention mechanism can comprise a vision transformer. In some embodiments, the vision transformer can comprise a natural language model. In some cases, the vision transformer can comprise a masked autoencoder, a Swin transformer, a vector quantized variational autoencoder, or another type of vision transformer. In some embodiments, the vision transformer can be combined with a generative adversarial network.

In some embodiments, the pooling layers can comprise global pooling layers. In some embodiments, the global pooling layers may combine the outputs of neuron clusters at one layer into a single neuron in the next layer. For example, max pooling layers may use the maximum value from each of a cluster of neurons in the prior layer; and average pooling layers may use the average value from each of a cluster of neurons at the prior layer.

In some embodiments, the fully-connected layers can connect every neuron in one layer to every neuron in another layer. In some cases in neural networks, each neuron may receive input from some number locations in the previous layer. In some cases, in a fully-connected layer, each neuron may receive input from every element of the previous layer.

In some embodiments, the normalization layer can be a batch normalization layer. In some embodiments, the batch normalization layer may improve the performance and stability of neural networks. In some embodiments, the batch normalization layer may provide any layer in a neural network with inputs that are zero mean/unit variance. In some cases, the advantages of using batch normalization layer may include faster trained networks, higher learning rates, easier to initialize weights, more activation functions viable, and simpler process of creating deep networks.

In some embodiments, the trained algorithm may be configured to accept a plurality of input variables and to produce one or more output values based on the plurality of input variables. In some embodiments, the trained algorithm may comprise a classifier, such that each of the one or more output values may comprise one of a fixed number of possible values. In some cases, the possible values can comprise a linear classifier, a logistic regression classifier. In some cases, the algorithm can indicate a classification of the biological sample or the subject by the classifier, or both. In some embodiments, the trained algorithm may comprise a binary classifier. In some embodiments, each of the one or more output values can comprise one of two values. In some embodiments, the values can comprise {0, 1}, {positive, negative}, or {high-risk, low-risk}, indicating a classification of the biological sample or subject, or both, by the classifier. In some embodiments, the trained algorithm may be another type of classifier, such that each of the one or more output values can comprise one of more than two values. In some embodiments, the values can comprise {0, 1, 2}, {positive, negative, or indeterminate}, or {high-risk, intermediate-risk, or low-risk}, indicating a classification of the biological sample or subject, or both, by the classifier. In some embodiments, the output values may comprise descriptive labels, numerical values, or a combination thereof. In some embodiments, some of the output values may comprise descriptive labels. In some embodiments, some of the output values may comprise numerical values, such as binary, integer, or continuous values. Such binary output values may comprise, for example, {0, 1}, {positive, negative}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a prognosis of the cancer-related category of the subject. Some numerical values may be mapped to descriptive labels, for example, by mapping 1 to “positive” and 0 to “negative.”

In some embodiments, the classification of samples may assign an output value of “indeterminate” or 2 if the sample is not classified as “positive,” “negative,” 1, or 0. In some cases, a set of two cutoff values can be used to classify samples into one of the three possible output values. Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. In some embodiments, sets of cutoff values may be used to classify samples into one of n+1 possible output values, where n is any positive integer.

### Visualization

In some embodiments, the systems, media, and methods disclosed herein can further comprise generating a data summary, result, or visualization, or any combination thereof. In some embodiments, visualization displays may comprise graphs, plots, and overlay points representing transcripts or cell annotations onto tissue images. In some embodiments, visualization may query that Seurat/tileDB object to retrieve the relevant data from the object for display. In some further embodiments, the relevant data may comprise transcript locations and/or cell annotations. In some embodiments, visualization may be displayed as transcript locations as points, cell annotations (e.g., cell type) as points, boxplots, violin plots, dot plots, or any combination thereof. In some embodiments, visualization on cell segmentation statistics may be displayed as points for raw values or projection coordinates in embedding space for evaluation of the segmentation outcomes of interest with respect to either of the alternative segmentation outcomes of same sample. In some embodiments, the visualization may form overlays between segmentation boundaries, transcripts and downstream data analysis output. In some cases, downstream data analysis output comprises cell types. In some cases, downstream data analysis output comprises one or more of cell states, phenotypes of tissue microenvironment, differential expression of cell type based on spatial context, quantifying subcellular expression, and spatially resolved biomarker identification, or any combination thereof. In some embodiments, the visualization may be from different segmentation configurations or methods, or the segmentation outcomes of reference samples.

In some embodiments, the visualization may be performed using a vision transformer. In some embodiments, the vision transformer can process an input image into a series of patches. In some cases, the vision transformer can serialize each patch into a vector. In some embodiments, the vision transformer can match each vector to a smaller dimension with matrix multiplication. In some embodiments, a transformer encoder can process the vision transformer data. In some embodiments, transformers can measure relationships between one or more input tokens through the transformer encoder. In some embodiments, the visualization may be trained in a masked encoder, a Swin transformer, a vector quantized variational autoencoder, or any combination thereof.

### EXAMPLES

The following illustrative examples are representative of embodiments of the software applications, systems, and methods described herein and are not meant to be limiting in any way.

### Example 1—Chromatic Corrections

The lateral chromatic aberration correction was performed on each multi-channel image using a pre-calibrated transform. As illustrated in FIG. 10, Raw Image on the left was applied to chromatic aberration and was corrected up. In correction, the fiduciary in all channels lining up was visibly observed in the Corrected Image on the right. The parameters of chromatic aberration correction are listed in Correction Transform table.

### Example 2—Spot Density Heatmap

As illustrated in FIGS. 13A and 13B, reasonable segmentation results were achieved and revealed cell structure that was not shown in original segmentation using the morphology markers alone. The 3 columns in FIG. 13A show morphology images of brain tissue with image channels including Histone stain, DAPI stain, and Soma rRNA stain. The 3 columns in FIG. 13B show readout density heatmap, segmentation based on all 3 morphology-stained images, and segmentation based on density heatmap & histone stain. The readout density heatmap in column I of FIG. 13B displays the contours of cell soma at higher signal-to-noise ratio with fine details as compared to the rRNA soma stain morphology image in the third column of FIG. 13A. As a result, the cell segmentation outcomes based on the combination of all 3 morphology-stained images and combination of the readout density heatmap and Histone stain are shown in the second and the third columns in FIG. 13B, respectively.

As illustrated in FIG. 24, image preprocessing techniques such as image sharpening and enhancement or machine-learning denoising models were applied to raw images to improve image quality. In one non-limiting example, FIG. 24 shows the raw image of readout density heatmap compared to the image restoration output after processing by cellpose 3.0 denoising model.

### Example 3—Comparison of 2-Channel and 5-Channel Model Outputs

FIG. 17A illustrates the initial network output using Cellpose algorithm. The gradient flow output was generated using the 2-channel model. Ground truth annotations were generated from segmentation results produced by existing custom cell segmentation algorithm, a process known as bootstrapping, based on the 2-channel model as initial ground truth. Subsequently, the annotations were reviewed and corrected to ensure quality of training data. The new 5-channel network model was initialized with the existing 2-channel model parameters, which helped reduce the training burden and yield good initial segmentation performance. The network was trained and fine-tuned to optimize the parameters for all channels based on the annotated ground truth. Parameter sets were finalized once they met a specified learning rate and minimize specific cost functions. FIG. 17B illustrates the output gradient flow result from the trained 5-channel model initialized from transfer learning. The trained and validated neural network can be exported to Open Neural Network Exchange Format (ONNX) model for interoperability across different frameworks and deployments.

### Example 4: Image Acquisition: HDR Settings

FIG. 25 illustrates a non-limiting example of an image acquired using High Definition Range (HDR) settings. In the non-limiting example of FIG. 25, a nuclei stained image was acquired with two non-HDR mode, one with a higher exposure time setting and one with a lower exposure time. In the non-limiting example shown in FIG. 25, DAPI nuclear staining was used and excited with UV at 385 nm and detected in the blue channel with emission at 512 nm. In the non-limiting example, the low exposure scan was acquired at 3 ms at 3% power (A) followed by 24 ms at 4% power (B). As shown in FIG. 25, some cells in B are saturated. As illustrated in FIG. 25, (C) shows the image acquired with HDR using both exposure times combined to provide a larger dynamic ranges and at the same time reduced intensity saturation. Illustrated in FIG. 25, (A) was an image acquired at 3 ms (low); (B) was an image acquired at 24 ms (high); and (C) was an image acquired with HDR.

For example, the morphology channels describing cell membrane used the following acquisition settings below in Table 2. In Table 2, the channel/dye used is listed, as well as the Excite/Emission wavelengths and the exposure times. For example, HDR with factor of 8 is used, meaning that two images per channel are acquired, one with the exposure time specified and one with one eighth exposure time compared to the initial image.

TABLE 2
Morphology Channels and Settings in Image Acquisition.

| | | Excitation | Emission | HDR Exposure |
|---|---|---|---|---|
| | Dye | (nm) | (nm) | Times (ms) |
| B | Alexa488 | 488 | 512 | 33/4 at 100% power |
| G | Atto532 | 532 | 553 | 25/3 at 100% power |
| Y | Dyomics605 | 590 | 630 | 100/12 at 100% |
| | | | | power |
| R | Alexa647 | 647 | 684 | 50/6 at 100% power |
| U | DAPI | 385 | 512 | 24/3 at 3% power |

### Example 5—Image Preprocessing: Nearest Neighbor Deconvolution

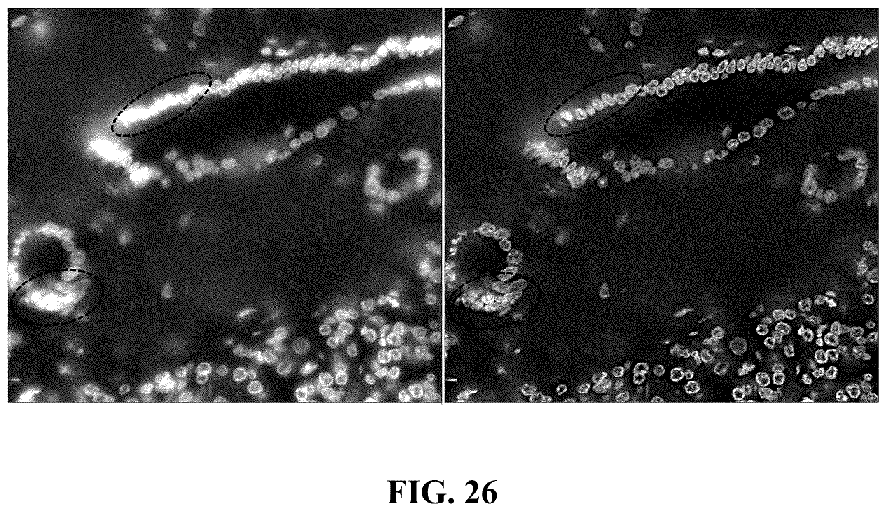

FIG. 26 illustrates a non-limiting example of output produced by the nearest neighbor (NN) deconvolution method. In FIG. 26, for example, a Gaussian filter sigma value of 9.7 pixels was used based on the previously described estimation, and the images were sampled at 800 nm in each z acquisition. For example, a multiplier A=0.75 was used to ensure that dim cells were still retained while removing majority of the blur signal. As illustrated in FIG. 26, areas are circled to mark regions where cells previously blurred by an out of focus signal (left panel) were recovered with the nearest neighbor deconvolution, uncovering clear boundaries between cells (right panel) that were previously difficult to see.

### Example 6—Intersection Over Union (IoU) Analysis

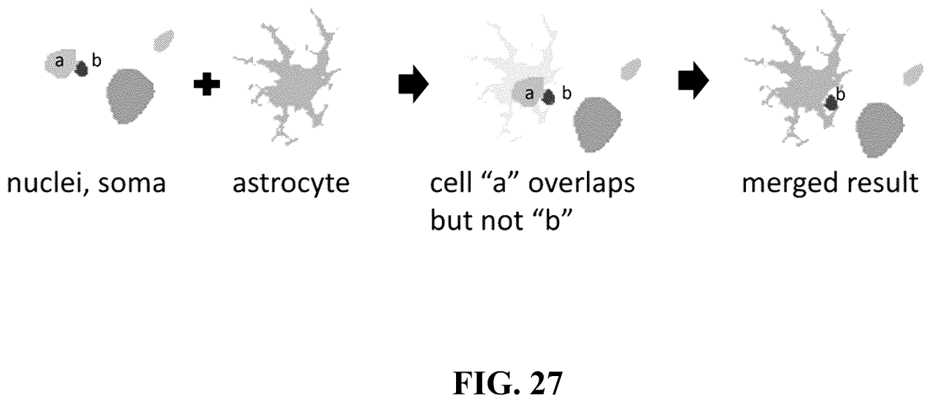

As illustrated in, for example, FIG. 27, improved segmentation was accomplished by integrating multiple segmentation steps from various modalities. For example, in some embodiments, combining segmentation of nuclei alone with segmentation of cytoplasm and nuclei can yield enhanced outcomes. For example, certain cells may lack adequate membrane staining, relying solely on nuclei staining, and vice versa. In some embodiments, this multi-modality segmentation approach can be used for tracing brain cells. In some embodiments, distinct segmentation steps can be needed to identify elongated protrusions, which are then integrated with nucleus and cell soma data. In some embodiments, the criteria for merging or splitting are determined through calculations involving intersection over union and area measurements, typically with a minimum overlap set. FIG. 27 illustrates a non-limiting example of cell protrusion merging.

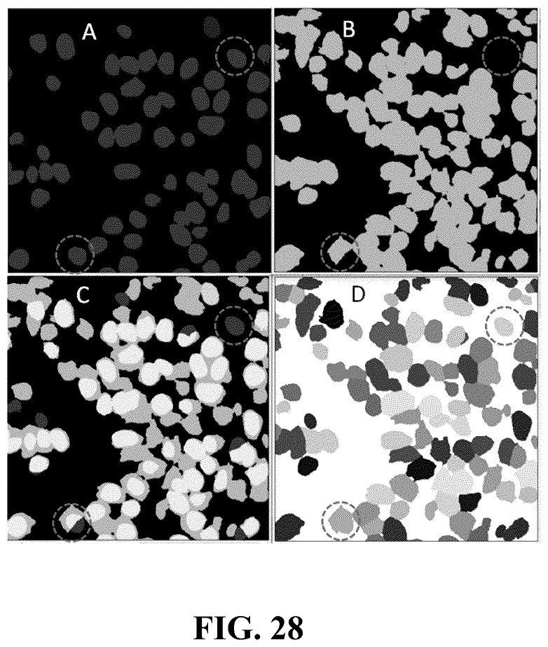

FIG. 28 illustrates a non-limiting example of IoU merging results between a nuclei segmentation output and a membrane plus nuclei segmentation. Areas encircled in FIG. 28 mark cases of cell merging. One merging example at the top right location of FIG. 28 shows that the cell mask was only detected in the nuclei segmentation step (A) and not at the cytoplasm/membrane segmentation step (B). The second merging example at the bottom left of FIG. 28 shows that when the two masks in (A) (B) overlap, the masks were merged, retaining the shapes from both (A) and (B)

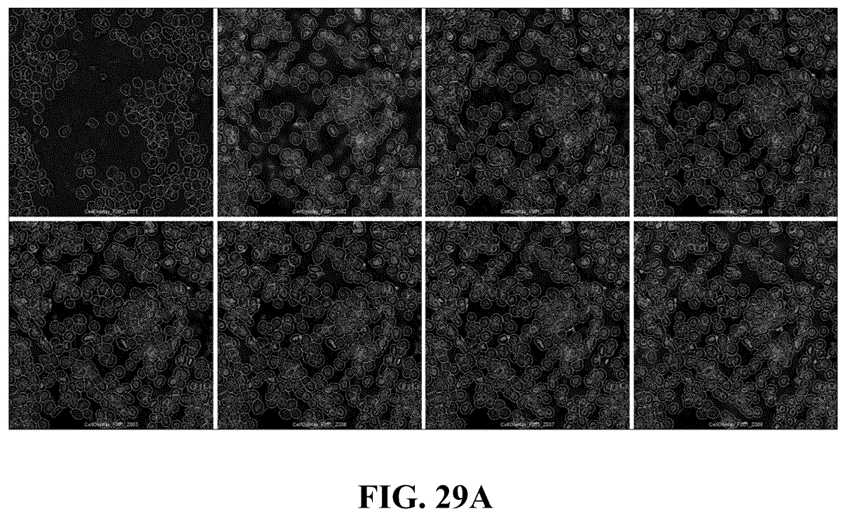

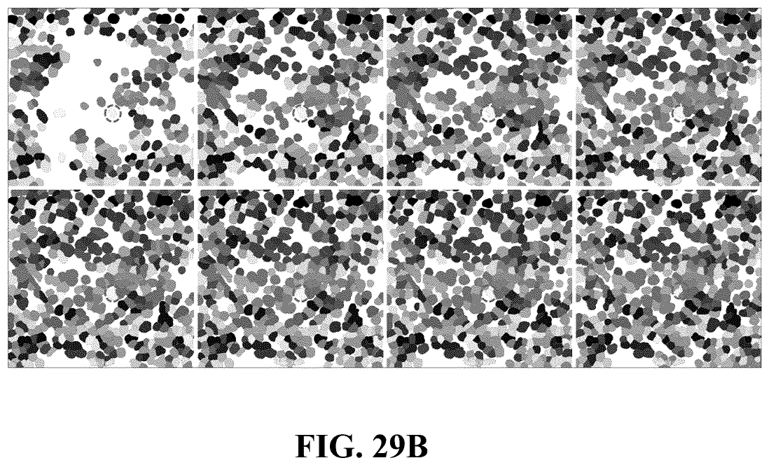

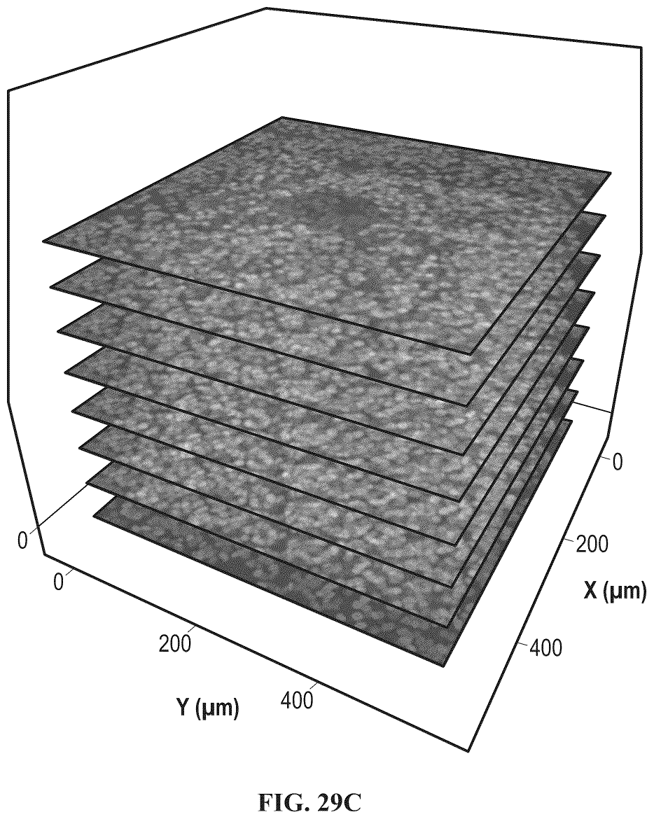

Similarly, as illustrated in FIGS. 29A-29C, this intersection analysis can be extended to correlate cells in different z-slices to perform 3D cell segmentation. FIG. 29A illustrates a non-limiting example of IoU merging results to correlate cellIds between multiple 2D image slices. FIG. 29B illustrates a non-limiting example of IoU merging results within a 3D volume. FIG. 29C shows a non-limiting example of one cell marked in all z-slices to show that this cell is segmented throughout the entire Z-stack. This data was generated using the NanoString whole transcriptome (Wtx) Pancreas public dataset.

### Example 7—Segmentation Results Across Different Tissue Types

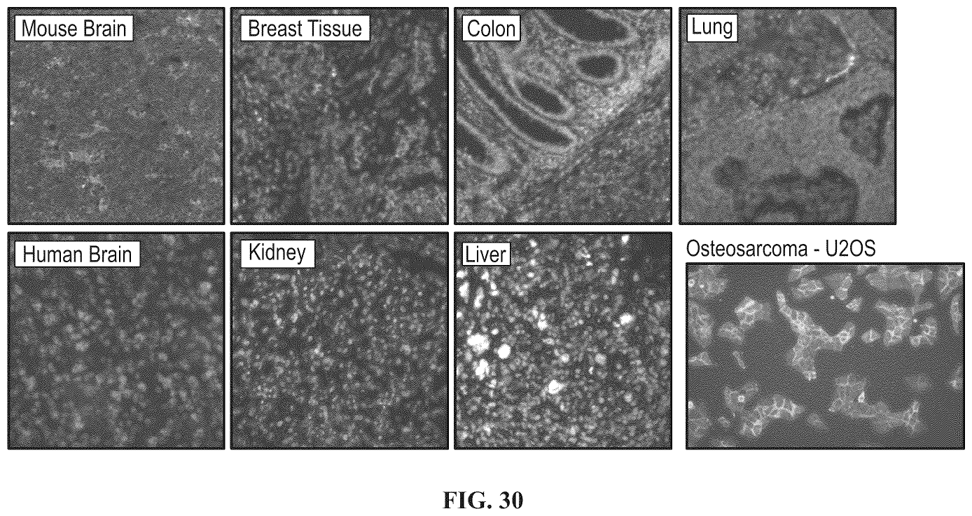

The multi-modal segmentation system has been tested in multiple tissue samples including those typically used in immune oncology research such as human breast, colon, lung, kidney, liver, and tonsils both in normal and diseased states. As illustrated in FIG. 30, it has successfully segmented human as well as mouse brain tissues and other tissues such as ovary, osteosarcoma, skin, and muscles.

### Example 8—Segmentation Mask Measurement Statistics Table

Using the segmentation masks, various measurements for the cells were be generated, including intensity and shape measurements. Tables 3 and 4 below show examples of the intensity and shape properties of a subset of cell populations, for example 20 out of 5309 cells. In some embodiments, hundreds of images can be acquired.

TABLE 3
Intensity and Shape Properties of Cellular Subset.

| | | | | | Mean- | Max- |
|---|---|---|---|---|---|---|
| CellId | CenterX | CenterY | Width | Height | DAPI | DAPI |
| 1 | 38 | 26 | 77 | 54 | 482 | 1392 |
| 2 | 218 | 32 | 85 | 65 | 424 | 1296 |
| 3 | 563 | 24 | 91 | 49 | 873 | 1984 |
| 4 | 684 | 15 | 76 | 32 | 512 | 1392 |
| 5 | 793 | 18 | 70 | 37 | 456 | 1568 |
| 6 | 1052 | 23 | 60 | 48 | 916 | 2224 |
| 7 | 1123 | 23 | 79 | 48 | 925 | 1936 |
| 8 | 1266 | 20 | 72 | 42 | 909 | 1808 |
| 9 | 1571 | 31 | 59 | 64 | 807 | 1632 |
| 10 | 1613 | 23 | 45 | 48 | 1147 | 1888 |
| 11 | 1663 | 19 | 76 | 40 | 1065 | 2032 |
| 12 | 1754 | 25 | 55 | 52 | 1449 | 2352 |
| 13 | 1804 | 16 | 61 | 34 | 985 | 2496 |
| 14 | 1995 | 25 | 71 | 51 | 618 | 1616 |
| 15 | 2083 | 19 | 69 | 39 | 394 | 1120 |
| 16 | 2149 | 21 | 68 | 43 | 432 | 1008 |
| 17 | 2215 | 25 | 81 | 51 | 510 | 1408 |
| 18 | 2393 | 20 | 80 | 41 | 391 | 1408 |
| 19 | 2518 | 29 | 61 | 60 | 1191 | 2496 |
| 20 | 2602 | 30 | 64 | 62 | | |

TABLE 4
Area and Shape Properties of Cellular Subset.

| Area | NucArea | AspectRatio | NucAspectRatio | Circularity | Compact [symbol] | Convexity | Eccentricit [symbol] | Perimeter | Solidity |
|---|---|---|---|---|---|---|---|---|---|
| 3607 | 2092 | 0.7 | 0.85 | 0.89 | 0.84 | 0.97 | 0.68 | 232 | 16 |
| 4442 | 2476 | 0.76 | 0.87 | 0.9 | 0.83 | 0.96 | 0.75 | 259 | 17.82 |
| 3440 | 1536 | 0.54 | 0.38 | 0.82 | 0.76 | 0.96 | 0.53 | 238 | 14.95 |
| 2122 | 1252 | 0.42 | 0.62 | 0.72 | 0.68 | 0.97 | 0.38 | 198 | 10.99 |
| 2014 | 0 | 0.53 | 0 | 0.78 | 0.74 | 0.97 | 0.53 | 185 | 11.19 |
| 2170 | 0 | 0.8 | 0 | 0.83 | 0.77 | 0.96 | 0.75 | 187 | 12 |
| 2669 | 1176 | 0.61 | 0.68 | 0.76 | 0.67 | 0.94 | 0.6 | 223 | 12.73 |
| 2318 | 988 | 0.58 | 1 | 0.79 | 0.68 | 0.93 | 0.58 | 206 | 12.07 |
| 2932 | 1120 | 0.92 | 0.71 | 0.89 | 0.75 | 0.92 | 0.84 | 221 | 14.44 |
| 1484 | 0 | 0.94 | 0 | 0.82 | 0.65 | 0.89 | 0.75 | 169 | 9.86 |
| 2104 | 0 | 0.53 | 0 | 0.73 | 0.6 | 0.91 | 0.51 | 209 | 11.07 |
| 1961 | 0 | 0.95 | 0 | 0.81 | 0.67 | 0.91 | 0.91 | 191 | 11.24 |
| 1707 | 0 | 0.56 | 0 | 0.8 | 0.72 | 0.95 | 0.53 | 172 | 10.43 |
| 2860 | 0 | 0.72 | 0 | 0.9 | 0.83 | 0.96 | 0.69 | 208 | 14.3 |
| 1940 | 1064 | 0.57 | 0.65 | 0.72 | 0.67 | 0.96 | 0.51 | 191 | 10.54 |
| 2325 | 1492 | 0.63 | 0.45 | 0.82 | 0.74 | 0.95 | 0.61 | 198 | 12.32 |
| 2863 | 1708 | 0.63 | 0.65 | 0.76 | 0.67 | 0.93 | 0.64 | 232 | 13.18 |
| 2584 | 1200 | 0.51 | 0.48 | 0.78 | 0.73 | 0.97 | 0.5 | 211 | 12.63 |
| 2469 | 1432 | 0.98 | 0.87 | 0.8 | 0.62 | 0.88 | 0.87 | 223 | 12.5 |

[symbol]

indicates data missing or illegible when filed

In some embodiments, in addition to the location, intensity and shape properties, this cell segmentation measurement can also include, for example, other properties such as cell fluorescent texture/entropy and neighborhood/cell-to-cell analysis. For example, to mark cells that are split across multiple FOVs, a calculation was performed by finding all cellids that intersect with FOV boundaries. Additionally, for these split cells, the ratio between these cells to the local average was measured. In some embodiments, if only a fraction of the cell segment is present in the current FOV, it can be further filtered out during analysis. Table 5 illustrates an example of the split cell measurements. In Table 5, values close to 1 indicate that those cells at the boundary may be retained since their size is similar to the average and only a small portion of the split cells are located at the neighboring FOVs.

TABLE 5
Split Ratio of Cells.
SplitRatioToLocal

1.16
1.42
1.1
0.68
0.65
0.7
0.85
0.74
0.94
0.48
0.67
0.63
0.55
0.92
0.62
0.74
0.92
0.83
0.79

1.-41. (canceled)

42. A computer-implemented system comprising a computing device comprising at least one processor and instructions executable by the at least one processor to provide a multi-modal segmentation application comprising:
(a) a software module configured to retrieve a 3D scan image of a biological sample in High Dynamic Range (HDR) mode, wherein the biological sample is labeled with one of a plurality of morphology markers;
(b) at least one of a software module configured to reduce the 3D scan image to a 2D image, wherein an optimal focus region in a z-slice is obtained; or a software module configured to deconvolve each of the 2D images to obtain an optimal focus region utilizing a 3D scan image stack of the 2D images;
(c) a software module configured to perform image preprocessing to correct and enhance images prior to segmentation, wherein the software module is further configured to perform subcellular segmentation on the biological sample based on the plurality of morphology markers;
(d) a software module configured to combine different imaging modalities, wherein the imaging modalities describe nucleic, cytoplasmic and membrane modalities;
(e) a software module configured to perform cell segmentation in 2D, 3D and time lapse images; and
(f) a software module configured to generate per cell properties and measurements of one or more of the images of (c)-(e), including fluorescent intensities and shape information as part of the processing results.

43. The system of claim 42, further comprising a software module configured to retrieve a readout density map from transcriptomic assays of the biological sample.

44. The system of claim 42, wherein the images are applied to a bleaching correction and deconvolution.

45. The system of claim 42, wherein the transcriptomic assays comprise gene expression assays with fluorescently labeled probes, RNA sequencing (RNA-seq), microarray analysis, reverse transcription polymerase chain reaction (RT-PCR), cap analysis of gene expression, or single-cell RNA sequencing (scRNA-seq), or any combination thereof.

46. The system of claim 42, wherein the images are applied to a chromatic aberration correction.

47. The system of claim 42, wherein subcellular segmentation comprises nucleic segmentation, cytoplasm segmentation, or extra-cellular segmentation.

48. The system of claim 42, wherein subcellular segmentation comprises training a machine learning algorithm and/or applying a machine learning algorithm.

49. A non-transitory computer-readable storage media encoded with instructions executable by one or more processors to provide a multi-modal segmentation application comprising:
(a) a software module configured to retrieve a 3D scan image of a biological sample in High Dynamic Range (HDR) mode, wherein the biological sample is labeled with one of a plurality of morphology markers;
(b) a software module configured to reduce the 3D scan image to a 2D image, wherein an optimal focus region in z-slice is obtained; alternatively, a software module configured to deconvolve the 2D images for to obtain an optimal focus region while maintaining z positions of the 2D images in a 3D scan image stack of the 2D images; and
(c) a software module configured to perform subcellular segmentation on the biological sample based on the plurality of morphology markers.

50. The non-transitory computer-readable storage media of claim 49, further comprising a software module configured to retrieve a readout density map from transcriptomic assays of the biological sample.

51. The non-transitory computer-readable storage media of claim 49, wherein the images are applied to a bleaching correction.

52. The non-transitory computer-readable storage media of claim 49, wherein the transcriptomic assays comprise gene expression assays with fluorescently labeled probes, RNA sequencing (RNA-seq), microarray analysis, reverse transcription polymerase chain reaction (RT-PCR), cap analysis of gene expression, or single-cell RNA sequencing (scRNA-seq), or any combination thereof.

53. The non-transitory computer-readable storage media of claim 49, wherein subcellular segmentation comprises nucleic segmentation, cytoplasm segmentation, or extra-cellular segmentation.

54. The non-transitory computer-readable storage media of claim 49, wherein subcellular segmentation comprises training a machine learning algorithm and/or applying a machine learning algorithm.

55. A computer-implemented method comprising:
(a) retrieving, by a computer, a 3D scan image of a biological sample in High Dynamic Range (HDR) mode, wherein the biological sample is labeled with one of a plurality of morphology markers;
(b) reducing the 3D scan image to a 2D image, wherein an optimal focus region in a z-slice is obtained; alternatively, performing deconvolution thereby creating the 2D images for an optimal contrast while maintaining z positions of the 2D images within a stack of the corresponding 3D scan images; and
(c) performing segmentation on the biological sample based on the plurality of morphology markers.

56. The method of claim 55, further comprising retrieving a readout density map from transcriptomic assays of the biological sample.

57. The method of claim 55, wherein the images are applied to a bleaching correction.

58. The method of claim 55, wherein the transcriptomic assays comprise gene expression assays with fluorescently labeled probes, RNA sequencing (RNA-seq), microarray analysis, reverse transcription polymerase chain reaction (RT-PCR), cap analysis of gene expression, or single-cell RNA sequencing (scRNA-seq), or any combination thereof.

59. The method of claim 55, wherein one or more image results of the subcellular segmentation are combined.

60. The method of claim 55, wherein subcellular segmentation comprises nucleic segmentation, cytoplasm segmentation, or extra-cellular segmentation.

61. The method of claim 55, wherein subcellular segmentation comprises training a machine learning algorithm and/or applying a machine learning algorithm.
